(19) United States
(12) Patent Application Publication
(10) Pub. No.: US 2024/0327895 A1
(43) Pub. Date: Oct. 3, 2024

(54) COMPOSITION FOR STABILIZING CELL-FREE NUCLEIC ACID AND/OR EXOSOMAL NUCLEIC ACID IN BLOOD, AND AGENT AND KIT CONTAINING THE SAME

(71) Applicant: Industrial Technology Research Institute, Hsinchu (TW)

(72) Inventors: Tseng-Huang LIU, Kaohsiung City (TW); Chien-Ming HSU, Taipei City (TW); Chih-Hung LEE, New Taipei City (TW)

(73) Assignee: Industrial Technology Research Institute, Hsinchu (TW)

(21) Appl. No.: 18/400,228

(22) Filed: Dec. 29, 2023

(30) Foreign Application Priority Data
Dec. 30, 2022 (TW) 111150824

(51) Int. Cl. C12Q 1/6806

(52) U.S. Cl. CPC C12Q 1/6806

(57) ABSTRACT

A composition for stabilizing cell-free nucleic acid and/or exosomal nucleic acid in blood is provided. The composition for stabilizing cell-free nucleic acid and/or exosomal nucleic acid in blood is (a) a first composition or (b) a second composition. The first composition includes (i) an antiseptic component including an allantoin-formaldehyde condensation product; (ii) an enzyme inhibitory component including a first metal chelator and (iii) a metabolic inhibitory component including NaF or sodium azide. The second composition includes (i) an antiseptic component including an allantoin-formaldehyde condensate; (ii) an enzyme inhibiting component including a first metal chelator; (iii) a metabolic an inhibitory component including NaF or sodium azide; and (iv) an anticoagulant component including a second metal chelator.

### CROSS REFERENCE TO RELATED APPLICATIONS

This Application claims priority of Taiwan Patent Application No. 111150824, filed on Dec. 30, 2022, the entirety of which is incorporated by reference herein.

### REFERENCE TO AN ELECTRONIC SEQUENCE LISTING

The contents of the electronic sequence listing (9044B-P220068701-US_ST26_Seq_Listing.xml; Size: 5,320 bytes; and Date of Creation: Dec. 28, 2023) is herein incorporated by reference in its entirety.

### TECHNICAL FIELD

The present disclosure relates to the stabilization of cell-free nucleic acid and/or exosomal nucleic acid in blood, and in particular to compositions for stabilizing cell-free nucleic acid and/or exosomal nucleic acid in blood, as well as agents and kits containing the same.

### BACKGROUND

In the 1940s, scientists discovered the existence of cell-free nucleic acid in blood (mainly cell-free DNA (cfDNA) in blood). Normal cells in the human body will release DNA free into the blood when they are metabolized in the body, and become cell-free DNA in blood. In clinical specimens, the sampling method for liquid biopsy is low-invasive (i.e. blood collection), which has the advantages of convenient sampling and instant detection and analysis, and cell-free DNA in blood is currently an important test sample for non-invasive prenatal diagnosis and tumor diagnosis.

However, cell-free DNA in blood degrades easily due to its short fragment length and small quantity, and the sampling, transportation, and storage processes may also cause nucleated cells in the blood to rupture and release genomic DNA, thereby interfering with the accuracy of subsequent testing. Therefore, it is usually recommended that subsequent processing, such as nucleic acid extraction, be performed immediately within 8 hours after collecting whole blood, and this results in limitations to the convenience of clinical diagnostic applications of cell-free DNA in blood.

Although preservation solutions specialized for free cell-DNA in blood are currently available to reduce the degradation of cell-free DNA in blood and maintain the integrity of other cells, most of them are not capable of preserving blood for long periods of time while maintaining good quality of cell-free nucleic acid and exosomal nucleic acid in blood.

Therefore, there is still an urgent need for a composition that can achieve the effects of preserving blood for a long time and stabilizing cell-free nucleic acid and/or exosomal nucleic acid in blood.

### SUMMARY

The present disclosure provides a composition for stabilizing cell-free nucleic acid and/or exosomal nucleic acid in blood. The composition for stabilizing cell-free nucleic acid and/or exosomal nucleic acid in blood is selected from a group consisting of the following compositions (a) and (b): (a) a first composition and (b) a second composition. The first composition comprises: (i) an antiseptic component, comprising: an allantoin-formaldehyde condensation product; (ii) an enzyme inhibitory component, comprising: a first metal chelator; and (iii) a metabolic inhibitory component, comprising: NaF or sodium azide, wherein a weight ratio of the antiseptic component, the enzyme inhibitory component and the metabolic inhibitory component is 1-3:0.001-0.05:0.005-0.08. The second composition comprises: (i) an antiseptic component, comprising: an allantoin-formaldehyde condensation product; (ii) an enzyme inhibitory component, comprising: a first metal chelator; (iii) a metabolic inhibitory component, comprising: NaF or sodium azide; and (iv) an anticoagulant component, comprising: a second metal chelator, wherein a weight ratio of the antiseptic component, the enzyme inhibitory component, the metabolic inhibitory component and the anticoagulant component is 1-3:0.001-0.05:0.1-2:0.5-8.

The present disclosure further provides a stabilizing agent for cell-free nucleic acid and/or exosomal nucleic acid in blood. The stabilizing agent for cell-free nucleic acid and/or exosomal nucleic acid in blood is selected from a group consisting of the following agent. (a) and (b): (a) a first agent and (b) a second agent. The first agent comprises: (i) an antiseptic component, comprising: an allantoin-formaldehyde condensation product; (ii) an enzyme inhibitory component, comprising: a first metal chelator; (iii) a metabolic inhibitory component, comprising: NaF or sodium azide; and (iv) a solvent, wherein in the first agent, a content of the antiseptic component is 5-15% (w/v), a content of the enzyme inhibitory component is 0.005-0.25% (w/v), and a content of the metabolic inhibitory component is 0.025-0.4% (w/v). The second agent comprises: (i) an antiseptic component, comprising: an allantoin-formaldehyde condensation product; (ii) an enzyme inhibitory component, comprising: a first metal chelator; (iii) a metabolic inhibitory component, comprising: NaF or sodium azide; (iv) an anticoagulant component, comprising: a second metal chelator; and (v) a solvent, wherein in the second agent, a content of the antiseptic component is 1-3% (w/v), a content of the enzyme inhibitory component is 0.001-0.05% (w/v), a content of the metabolic inhibitory component is 0.1-2% (w/v), and a content of the anticoagulant component is 0.5-8% (w/v).

The present disclosure also provides a collection kit for cell-free nucleic acid and/or exosomal nucleic acid in blood, comprising the stabilizing agent for cell-free nucleic acid and/or exosomal nucleic acid in blood mentioned above; and a first container for accommodating a blood sample.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows a manner of mixing whole blood and a stabilizing agent;

FIG. 2 shows the hemolysis observation results of Sample 1 to Sample 3 and Sample 5 to Sample 9, as well as the Qiagen control sample, the Streck control sample and the EDTA control sample (whole blood is collected solely through an EDTA blood collection tube) in the efficacy test of stabilizing agents containing different concentrations of diazolidinyl urea (DU);

FIG. 3 shows the real-time polymerase chain reaction results of cell-free DNA in blood of Sample 1 to Sample 3 and Sample 5 to Sample 9, as well as the Qiagen control sample, the Streck control sample and the EDTA control sample (whole blood is collected solely through an EDTA blood collection tube) (whole blood is collected solely through an EDTA blood collection tube) in the efficacy test of stabilizing agents containing different concentrations of diazolidinyl urea;

FIG. 4A shows the capillary electrophoresis result of cell-free DNA in blood of Sample 1 in the efficacy test of stabilizing agents containing different concentrations of diazolidinyl urea;

FIG. 4B shows the capillary electrophoresis result of cell-free DNA in blood of Sample 2 in the efficacy test of stabilizing agents containing different concentrations of diazolidinyl urea;

FIG. 4C shows the capillary electrophoresis result of cell-free DNA in blood of Sample 3 in the efficacy test of stabilizing agents containing different concentrations of diazolidinyl urea;

FIG. 4D shows the capillary electrophoresis result of cell-free DNA in blood of Sample 5 in the efficacy test of stabilizing agents containing different concentrations of diazolidinyl urea;

FIG. 4E shows the capillary electrophoresis result of cell-free DNA in blood of Sample 6 in the efficacy test of stabilizing agents containing different concentrations of diazolidinyl urea;

FIG. 4F shows the capillary electrophoresis result of cell-free DNA in blood of Sample 7 in the efficacy test of stabilizing agents containing different concentrations of diazolidinyl urea;

FIG. 4G shows the capillary electrophoresis result of cell-free DNA in blood of Sample 8 in the efficacy test of stabilizing agents containing different concentrations of diazolidinyl urea;

FIG. 4H shows the capillary electrophoresis result of cell-free DNA in blood of Samples 9 in the efficacy test of stabilizing agents containing different concentrations of diazolidinyl urea;

FIG. 4I shows the capillary electrophoresis result of cell-free DNA in blood of the Qiagen control sample in the efficacy test of stabilizing agents containing different concentrations of diazolidinyl urea;

FIG. 4J shows the capillary electrophoresis result of cell-free DNA in blood of the Streck control sample in the efficacy test of stabilizing agents containing different concentrations of diazolidinyl urea;

FIG. 4K shows the capillary electrophoresis result of cell-free DNA in blood of the EDTA control sample (whole blood is collected solely through an EDTA blood collection tube) (whole blood is collected solely through an EDTA blood collection tube) in the efficacy test of stabilizing agents containing different concentrations of diazolidinyl urea;

FIG. 5 shows the hemolysis observation results of Samples D1 to Sample D9, as well as the Qiagen control sample and the Streck control sample in the efficacy test of stabilizing agents containing different concentrations of diazolidinyl urea (whole blood is collected through EDTA blood collection tubes);

FIG. 6 shows the real-time polymerase chain reaction results of cell-free DNA in blood of Samples D1 to Sample D9, as well as the Qiagen control sample and the Streck control sample in the efficacy test of stabilizing agents containing different concentrations of diazolidinyl urea (whole blood is collected through EDTA blood collection tubes);

FIG. 7A shows the capillary electrophoresis result of cell-free DNA in blood of Sample D2 in the efficacy test of stabilizing agents containing different concentrations of diazolidinyl urea (whole blood is collected through EDTA blood collection tubes);

FIG. 7B shows the capillary electrophoresis result of cell-free DNA in blood of Samples D3 in the efficacy test of stabilizing agents containing different concentrations of diazolidinyl urea (whole blood is collected through EDTA blood collection tubes);

FIG. 7C shows the capillary electrophoresis result of cell-free DNA in blood of Sample D4 in the efficacy test of stabilizing agents containing different concentrations of diazolidinyl urea (whole blood is collected through EDTA blood collection tubes);

FIG. 7D shows the capillary electrophoresis result of cell-free DNA in blood of Sample D5 in the efficacy test of stabilizing agents containing different concentrations of diazolidinyl urea (whole blood is collected through EDTA blood collection tubes);

FIG. 7E shows the capillary electrophoresis result of cell-free DNA in blood of Sample D6 in the efficacy test of stabilizing agents containing different concentrations of diazolidinyl urea (whole blood is collected through EDTA blood collection tubes);

FIG. 7F shows the capillary electrophoresis result of cell-free DNA in blood of Sample D7 in the efficacy test of stabilizing agents containing different concentrations of diazolidinyl urea (whole blood is collected through EDTA blood collection tubes):

FIG. 7G shows the capillary electrophoresis result of cell-free DNA in blood of Samples D8 in the efficacy test of stabilizing agents containing different concentrations of diazolidinyl urea (whole blood is collected through EDTA blood collection tubes);

FIG. 7H shows the capillary electrophoresis result of cell-free DNA in blood of Sample D9 in the efficacy test of stabilizing agents containing different concentrations of diazolidinyl urea (whole blood is collected through EDTA blood collection tubes);

FIG. 7I shows the capillary electrophoresis result of cell-free DNA in blood of the Qiagen control sample in the efficacy test of stabilizing agents containing different concentrations of diazolidinyl urea (whole blood is collected through EDTA blood collection tubes);

FIG. 7J shows the capillary electrophoresis result of cell-free DNA in blood of Streck control sample in the efficacy test of stabilizing agents containing different concentrations of diazolidinyl urea (whole blood is collected through EDTA blood collection tubes);

FIG. 8 shows the hemolysis observation results of Sample E1 to Sample E9, as well as the Qiagen control sample, the Streck control sample and the EDTA control sample (whole blood is collected solely through a EDTA blood collection tube) in the efficacy test of stabilizing agents containing different concentrations of EDTA;

FIG. 9 shows the real-time polymerase chain reaction results of cell-free DNA in blood of Sample E1 to Sample E9 in the efficacy test of stabilizing agents containing different concentrations of EDTA;

FIG. 10A shows the capillary electrophoresis result of cell-free DNA in blood of Sample E1 in the efficacy test of stabilizing agents containing different concentrations of EDTA;

FIG. 10B shows the capillary electrophoresis result of cell-free DNA in blood of Sample E2 in the efficacy test of stabilizing agents containing different concentrations of EDTA;

FIG. 10C shows the capillary electrophoresis result of cell-free DNA in blood of Sample E4 in the efficacy test of stabilizing agents containing different concentrations of EDTA;

FIG. 10D shows the capillary electrophoresis result of cell-free DNA in blood of Sample E5 in the efficacy test of stabilizing agents containing different concentrations of EDTA;

FIG. 10E shows the capillary electrophoresis result of cell-free DNA in blood of Sample E6 in the efficacy test of stabilizing agents containing different concentrations of EDTA;

FIG. 10F shows the capillary electrophoresis result of cell-free DNA in blood of Sample E7 in the efficacy test of stabilizing agents containing different concentrations of EDTA;

FIG. 10G shows the capillary electrophoresis result of cell-free DNA in blood of Sample E8 in the efficacy test of stabilizing agents containing different concentrations of EDTA;

FIG. 10H shows the capillary electrophoresis result of cell-free DNA in blood of Sample E9 in the efficacy test of stabilizing agents containing different concentrations of EDTA;

FIG. 10I shows the capillary electrophoresis result of cell-free DNA in blood of the Qiagen control sample in the efficacy test of stabilizing agents containing different concentrations of EDTA:

FIG. 10J shows the capillary electrophoresis result of cell-free DNA in blood of the Streck control sample in the efficacy test of stabilizing agents containing different concentrations of EDTA;

FIG. 10K shows the capillary electrophoresis result of cell-free DNA in blood of the EDTA control sample (whole blood is collected solely through a EDTA blood collection tube) in the efficacy test of stabilizing agents containing different concentrations of EDTA;

FIG. 11 shows the hemolysis observation results of the test samples 1 and the control samples in the long-term efficacy test of stabilizing agents;

FIG. 12 shows the real-time polymerase chain reaction results of cell-free DNA in blood of the test samples and the control samples on Day 0, Day 4, Day 7 and Day 14 after the mixing in the long-term efficacy test of stabilizing agents;

FIG. 13A shows the capillary electrophoresis result of cell-free DNA in blood of the test samples and the control samples on Day 0 after the mixing in the long-term efficacy test of stabilizing agents;

FIG. 13B shows the capillary electrophoresis result of cell-free DNA in blood of the test samples and the control samples on Day 4 after the mixing in the long-term efficacy test of stabilizing agents;

FIG. 13C shows the capillary electrophoresis result of cell-free DNA in blood of the test samples and the control samples on Day 7 after the mixing in the long-term efficacy test of stabilizing agents;

FIG. 13D shows the capillary electrophoresis result of cell-free DNA in blood of the test samples and the control samples on Day 14 after the mixing in the long-term efficacy test of stabilizing agents; and

FIG. 14 shows the reverse transcription real-time polymerase chain reaction results of exosomal free RNA in blood of the teat sample and the control sample on Day 4 and Day 7 after the mixing.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure may provide a composition for stabilizing cell-free nucleic acid and/or exosomal nucleic acid in blood.

The composition for stabilizing cell-free nucleic acid and/or exosomal nucleic acid in blood of the present disclosure can maintain the stability of the quality of cell-free nucleic acid and exosomal nucleic acid in whole blood to avoid their degradation, and can also maintain the integrity of white blood cells to avoid the release of genomic nucleic acids due to the rupture of white blood cells, thereby causing background interference in back-end molecular detection, and can reduce the occurrence of hemolysis. Through the use of the composition for stabilizing cell-free nucleic acid and/or exosomal nucleic acid in blood of the present disclosure, whole blood or plasma can be stored at room temperature for a long time, and the quality the cell-free nucleic acid and/or exosomal nucleic acid in whole blood or plasma can be effectively maintained.

The cell-free nucleic acid in blood described herein may include cell-free DNA (cfDNA) in blood, cell-free RNA (cfRNA) in blood, etc., or any combination thereof, but they are not limited thereto. In one embodiment, the aforementioned cell-free nucleic acid in blood is cell-free DNA in blood.

Moreover, the aforementioned cell-free DNA in blood may include, but is not limited to, circulating tumor DNA (ctDNA), cell-free fetal DNA (cffDNA), etc., or any combination thereof.

Furthermore, the exosomal nucleic acid mentioned herein may include DNA. RNA, etc. in exosomes, or any combination thereof, but it is not limited thereto.

Exosomal RNA may include messenger RNA (mRNA), microRNA (miRNA), long non-coding RNA (lncRNA), and competitive endogenous RNA (competing endogenous RNA, ceRNA), etc. in exosomes, or any combination thereof, but it is not limited to thereto.

The composition for stabilizing cell-free nucleic acid and/or exosomal nucleic acid in blood of the present disclosure is capable of maintaining cell-free nucleic acid and/or exosomal nucleic acid in blood for at least 4 days, such as at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, but it is not limited thereto.

Examples of the compositions for stabilizing cell-free nucleic acid and/or exosomal nucleic acid in blood of the present disclosure may include a first composition or a second composition described below, but they are not limited thereto.

The aforementioned first composition may include, but is not limited to, an antiseptic component, an enzyme inhibitory component and a metabolic inhibitory component.

The antiseptic component mentioned above may include, an allantoin-formaldehyde condensation product, but it is not limited thereto. The allantoin-formaldehyde condensation product may include, but is not limited to, diazolidinyl urea (DU), imidazolidinyl urea (IDU), etc., or any combination thereof. In one embodiment, the aforementioned allantoin-formaldehyde condensation product may be diazolidinyl urea.

The aforementioned enzyme inhibitory component may include, a first metal chelator, but it is not limited thereto. The aforementioned first metal chelator may include, but is not limited to, aurintricarboxylic acid (ATA), etc.

Moreover, the foregoing metabolic inhibitory component may include NaF, sodium aside, etc., or any combination thereof. In one embodiment, the foregoing metabolic inhibitory component may be NaF.

Furthermore, in one embodiment, in the aforementioned first composition, the weight ratio of the antiseptic component, the enzyme inhibitory component and the metabolic inhibitory component may be about 1-3:0.0001-0.05:0.005-0.08, such as about 1-2.5:0.0015-0.05:0.005-0.05, about 1-2:0.002-0.025:0.005-0.01, about 1-1.5:0.003-0.03:0.045-0.05, about 1-1.8:0.0032:0.06, about 1:0.0032:0.06, about 1.2:0.0032:0.06, about 1.4:0.0032:0.06, about 1.6:0.0032:0.06, about 1.8:0.0032:0.06, but it is not limited thereto.

In addition, in one embodiment, in the first composition mentioned above, the antiseptic component mentioned above may include allantoin-formaldehyde condensation product while the allantoin-formaldehyde condensation product may be diazolidinyl urea, the enzyme inhibitor mentioned above may include a first metal chelator while the first metal chelator may be aurintricarboxylic acid, and the metabolism inhibitory component mentioned above may be NaF. Moreover, in this embodiment, in the aforementioned first composition, the weight ratio of the antiseptic component, the enzyme inhibitory component and the metabolic inhibitory component may be about 1-3:0.001-0.05:0.005-0.08, such as about 1-2.5:0.0015-0.05:0.005-0.05, about 1-20.002-0.025:0.008-0.01, about 1-1.5:0.003-0.03:0.045-0.05, about 1-1.8:0.0032:0.06, about 1:0.0032:0.06, about 1.2:0.0032:0.06, about 1.4:0.0032:0.06, about 1.6:0.0032:0.06, about 1.8:0.0032:0.06, but it is not limited thereto. In one specific embodiment of this embodiment, in the first composition mentioned above, the weight ratio of the antiseptic component, the enzyme inhibitory component and the metabolic inhibitory component may be about 1-1.8:0.0032:0.06.

In one specific embodiment, the aforementioned first composition consists of the aforementioned antiseptic component, the aforementioned enzyme inhibitory component and the aforementioned metabolic inhibitory component while the aforementioned antiseptic component is diazolidinyl urea, the aforementioned enzyme inhibitor is aurintricarboxylic acid and the aforementioned metabolic inhibitory component is NaF. Furthermore, in this specific embodiment, in the aforementioned first composition, the weight ratio of the antiseptic component, the enzyme inhibitory component and the metabolic inhibitory component may be about 1-1.8:0.0032:0.06.

On the other hand, the aforementioned second composition may include, but is not limited to, an antiseptic component, an enzyme inhibitory component, a metabolic inhibitory component and an anticoagulant component.

The types and/or further examples of the antiseptic component, the enzyme inhibitory component and the metabolic inhibitory component in the second composition mentioned above may be the same as the types and/or further examples of the antiseptic component, the enzyme inhibitory component and the metabolic inhibitory component in the first composition mentioned above, and can be referred to the relevant description for the antiseptic component, the enzyme inhibitory component and the metabolic inhibitory component in the first composition mentioned above in the previous paragraphs, and thus will not be repeated herein.

Moreover, the anticoagulant component in the aforementioned second composition may include, but is not limited to, a second metal chelator, etc.

The foregoing second metal chelator may include, ethylenediaminetetraacetic acid (EDTA), citrate, etc., or any combination thereof, but it is not limited thereto. In one embodiment, the foregoing second metal chelator may be ethylenediaminetetraacetic acid.

In one embodiment, in the foregoing second composition, the weight ratio of the antiseptic component, the enzyme inhibitory component, the metabolic inhibitory component and the anticoagulant component may be about 1-3:0.001-0.050.1-2:0.5-8, such as about 1-2.5:0.005-0.05:0.2-1.8:0.8-6, about 1-2:0.008-0.04:0.3-1.5:1-5.8, about 1-1.8:0.01-0.03:0.4-1.2:1-5.6, about 1-1.8:0.016:0.3:1.2-5.8, about 1-1.8:0.016:0.3:1.2-5.8, about 1:0.016:0.3:2.6, about 1:0.016:0.3:2.8, about 1:0.016.0.3:3, about 1:0.016:0.3:3.2, about 1:0.016:0.3:3.4, about 1:0.016:0.3:3.6, about 1:0.016:0.3:3.8, about 1:0.016:0.3:4, about 1:0.016:0.3:4.8, about 1:0.016:0.3:5.8, about 1:0.016:0.3:1.2, about 1.2:0.016:0.3:1.2, 1.4:0.016:0.3:1.2, about 1.6:0.016:0.3:1.2, about 1.8:0.016:0.3:1.2, but it is not limited thereto.

Furthermore, in one embodiment, in the aforementioned second composition, the aforementioned antiseptic component may include allantoin-formaldehyde condensation product while the allantoin-formaldehyde condensation product may be diazolidinyl urea, the aforementioned enzyme inhibitor may include a first metal chelator while the first metal chelator may be aurintricarboxylic acid, the aforementioned metabolic inhibitory component may be NaF, and the aforementioned anticoagulant component may include a second metal chelator while the second metal chelator may be ethylenediaminetetraacetic acid. In addition, in this embodiment, in the aforementioned second composition, the weight ratio of the antiseptic component, the enzyme inhibitory component, the metabolic inhibitory component and the anticoagulant component may be about 1-3:0.001-0.05:0.1-2:0.5-8, such as about 1-2.5:0.005-0.05:0.2-1.8:0.8-6, about 1-2:0.008-0.04:0.3-1.5:1-5.8, about 1-1.8:0.01-0.03:0.4-1.2:1-5.6, about 1-1.8:0.016:0.31.2-5.8, about 1-1.8:0.016:0.3:1.2-5.8, about 1:0.016:0.3:2.6, about 1:0.016:0.3:2.8, about 1:0.016:0.3:3, about 1:0.016:0.3:3.2, about 1:0.016:0.3:3.4, about 1:0.016:0.3:3.6, about 1:0.016:0.3:3.8, about 1:0.016:0.3:4, about 1:0.016:0.3:4.8, about 1:0.016:0.3:5.8, about 1:0.016:0.3:1.2, about 1.2:0.016:0.3:1.2, 1.4:0.016:0.3:1.2, about 1.6:0.016:0.3:1.2, about 1.8:0.016:0.3:1.2, but it is not limited thereto. In one specific embodiment of this embodiment, the weight ratio of the antiseptic component, the enzyme inhibitory component, the metabolic inhibitory component and the anticoagulant component may be about 1-1.8:0.3:0.0016:1.2-5.8.

In one specific embodiment, the aforementioned second composition consists of the above-mentioned antiseptic component, the above-mentioned enzyme inhibitory component, the above-mentioned metabolic inhibitory component and the above-mentioned anticoagulant component while the above-mentioned antiseptic component is diazolidinyl urea, the above-mentioned enzyme inhibitor is aurintricarboxylic acid, the above-mentioned metabolic inhibitory component is NaF and the above-mentioned anticoagulant component is ethylenediaminetetraacetic acid. Furthermore, in this specific embodiment, the weight ratio of the above-mentioned antiseptic component, the above-mentioned enzyme inhibitory component, the above-mentioned metabolic inhibitory component and the above-mentioned anticoagulant component may be about 1-1.8:0.016:0.3:1.2-5.8.

In one embodiment, the composition for stabilizing cell-free nucleic acid and/or exosomal nucleic acid in blood of the present disclosure mentioned above may be any first compositions mentioned above. In another embodiment, the composition for stabilizing cell-free nucleic acid and/or exosomal nucleic acid in blood of the present disclosure mentioned above may be any second compositions mentioned above.

The aforementioned composition for stabilizing cell-free nucleic acid and/or exosomal nucleic acid in blood of the present disclosure may be further collocated with a solvent to form a stabilizing agent. By directly mixing whole blood or plasma with this stabilizing agent, the foregoing effects, such as maintaining the stability of the quality of cell-free nucleic acid and exosomal nucleic acid in whole blood, maintaining the integrity of white blood cells, and reducing the occurrence of hemolysis can be achieved.

The weight ratio of the foregoing composition for stabilizing cell-free nucleic acid and/or exosomal nucleic acid in blood of the present disclosure to the foregoing solvent may be about 1:4-65, such as about 1:4.5-03, about 1:5-62, about 1:5.5-61, about 1:6-6p, about 1:8-55, about 1:10-50, about 1:15-45, about 1:20-40, about 1:25-35, about 1:4, about 1:5, about 1:6, about 1:7, about 1:8, about 1:9, about 1:10, about 1:15, about 1:20, about 1:25, about 1:30, about 1:35, about 1:40, about 1:45, about 1:50, about 1:55, about 1:60, about 1:61, about 1:62, about 1:63, about 1:64, about 1:65.

Moreover, whole blood or plasma may be mixed with the aforementioned stabilizing agent at a volume ratio of about 1:0.05-0.5, for example, whole blood or plasma may be mixed with the aforementioned stabilizing agent at a volume ratio of about 1:0.1-0.4, about 1:0.15-0.3, about 1:0.2-0.25, about 1:0.05, about 1:0.1, about 1:0.15, about 1:0.2, about 1:0.25, about 1:0.3, about 1:0.35, about 1:0.4, about 1:0.45, about 1:0.5, but it is not limited thereto.

Accordingly, based on the above content, the present disclosure may further provide a stabilizing agent for cell-free nucleic acid and/or exosomal nucleic acid in blood.

The stabilizing agent for cell-free nucleic acid and/or exosomal nucleic acid in blood of the present disclosure is capable of maintaining cell-free nucleic acid and/or exosomal nucleic acid in blood for at least 4 days, such as at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, but it is not limited thereto.

Examples of the stabilizing agent for cell-free nucleic acid and/or exosomal nucleic acid in blood of the present disclosure may include a first agent or a second agent described below, but they are not limited thereto.

The aforementioned first agent may include, but is not limited to, an antiseptic component, an enzyme inhibitory component, a metabolic inhibitory component and a solvent.

The types and/or further examples of the antiseptic component, the enzyme inhibitory component and the metabolic inhibitory component in the first agent mentioned above may be the same as the types and/or further examples of the antiseptic component, the enzyme inhibitory component and the metabolic inhibitory component in the first composition mentioned above, and can be referred to the relevant description for the antiseptic component, the enzyme inhibitory component and the metabolic inhibitory component in the first composition mentioned above in the previous paragraphs, and thus will not be repeated herein.

The solvent in the aforementioned first agent may include water, such as distilled water, secondary water (deionized water), reverse osmosis water, ultra-pure grade water, but it is not limited thereto.

In the first agent mentioned above, the content of the antiseptic component mentioned above may be about 5-15% (w v), such as about 6-12% (w/v), about 8-10% (w/v), about 5% (w/v), about 6% (w/v), about 7% (w/v), about 8% (w/v), about 9% (w/v), about 10% (w/v), about 11% (w/v), about 12% (w/v), about 13% (w/v), about 14% (w/v), about 15% (w/v), but it is not limited thereto.

In the first agent mentioned above, the content of the metabolic inhibitory component mentioned above may be about 0.005-0.25% (w/v), such as about 0.006-0.24% (w/v), about 0.007-0.23% (w/v), about 0.008-0.22% (w/v), about 0.009-0.21% (w/v), about 0.01-0.2% (w/v), about 0.015-0.15% (w/v), about 0.016-0.1% (w/v), about 0.02-0.05% (w/v), about 0.005% (w/v), about 0.006% (w/v), about 0.007% (w/v), about 0.008% (w/v), about 0.009% (w/v), about 0.01% (w/v), about 0.012% (w/v), about 0.014% (w/v), about 0.015% (w/v), about 0.016% (w/v), about 0.0184% (w/v), about 0.02% (w/v), about 0.021% (w/v), about 0.022% (w/v), about 0.023% (w/v), about 0.024% (w/v), about 0.025% (w/v), about 0.03% (w/v), about 0.05% (w/v), about 0.08% (w/v), about 0.1% (w/v), about 0.15% (w/v), about 0.2% (w/v), about 0.25% (w/v), but it is not limited thereto.

In the first agent mentioned above, the content of the metabolic inhibitory component mentioned above may be about 0.025-0.4% (w/v), such as about 0.03-0.35% (w/v), about 0.032-0.3% (w/v), about (1.035-0.3% (w/v), about 0.038-0.25% (w/v), about 0.04-0.2% (w/v), about 0.045-0.15% (w/v), about 0.05-0.1% (w/v), about 0.025% (w/v), about 0.03% (w/v), about 0.04% (w/v), about 0.05% (w/v), about 0.06% (w/v), about 0.07% (w v), about 0.08% (w/v), about 0.09% (w/v), about 0.1% (w/v), about 0.15% (w/v), about 0.2% (w/v), about 0.25% (w/v), about 0.3% (w/v), about 0.35% (w/v), about 0.4% (w/v), but it is not limited thereto.

In one embodiment, in the first agent mentioned above, the content of the antiseptic component mentioned above may be about 5-15% (w/v), the content of the enzyme inhibitory component mentioned above may be about 0.005-0.25%% (w/v), and the content of the metabolic inhibitor mentioned above may be about 0.025-0.4% (w/v).

Moreover, in another embodiment, in the first agent mentioned above, the antiseptic component mentioned above may include allantoin-formaldehyde condensation product while the allantoin-formaldehyde condensation product may be diazolidinyl urea, the enzyme inhibitor mentioned above may include a first metal chelator while the first metal chelator may be aurintricarboxylic acid, the metabolic inhibitory component may be NaF, and the solvent mentioned above may be water. Furthermore, in this embodiment, in the first agent mentioned above, the content of the antiseptic component mentioned above may be about 5-15% (w/v), the content of enzyme inhibitory component mentioned above may be about 0.005-0.25% (w/v), and the content of the metabolic inhibitor mentioned above may be about 0.025-0.4% (w/v). In one specific embodiment of this embodiment, in the Cast agent mentioned above, the content of the antiseptic component mentioned above may be about 5-9% (w/v), the content of enzyme inhibitory component mentioned above may be about 0.016% (w/v), and the content of the metabolic inhibitor mentioned above may be about 0.3% (w/v).

In one specific embodiment, the aforementioned first agent consists of the aforementioned antiseptic component, the aforementioned enzyme inhibitory component, the aforementioned metabolic inhibitory component and the aforementioned solvent while the aforementioned antiseptic component is diazolidinyl urea, the aforementioned enzyme inhibitor is aurintricarboxylic acid, the aforementioned metabolic inhibitory component is NaF and the aforementioned solvent is water. Furthermore, in this specific embodiment, in the first agent, the content of the aforementioned antiseptic component may be about 5-9% (w/v), the content of the aforementioned enzyme inhibitory component may be about 0.016% (w/v), and the content of the aforementioned metabolic inhibitor may be about 0.3% (w/v).

On the other hand, the aforementioned second agent may include, but is not limited to, an antiseptic component, an enzyme inhibitory component, a metabolic inhibitory component, an anticoagulant component and a solvent.

The types and/or further examples of the antiseptic component, the enzyme inhibitory component, the metabolic inhibitory component and the anticoagulant component in the second agent mentioned above may be the same as the types and/or further examples of the antiseptic component, the enzyme inhibitory component, the metabolic inhibitory component and the anticoagulant component in the second composition mentioned above, and can be referred to the relevant description for the antiseptic component, the enzyme inhibitory component, the metabolic inhibitory component and the anticoagulant component in the second composition mentioned above in the previous paragraphs, and thus will not be repeated herein.

Furthermore, the types and/or further examples of the solvent in the second agent mentioned above may be the same as the types and/or further examples of the solvent in the first agent mentioned above, and can be referred to the relevant description for the solvent in the first agent mentioned above in the previous paragraphs, and thus will not be repeated herein.

In the aforementioned second agent, the content of the aforementioned antiseptic component may be about 1-3% (w/v), such as about 1.1-2.9% (w/v), about 1.2-2.8% (w/v), about 1.3-2.7% (w/v), about 1.4-2.6% (w/v), about 1.5-2.5% (w/v), about 1.6-2.4% (w/v), about 1% (w/v), about 1.2% (w/v), about 1.4% (w/v), about 1.6% (w/v), about 1.8% (w/v), about 2% (w/v), about 2.2% (w/v), about 2.4% (w/v), about 2.6% (w/v), about 2.8% (w/v), about 3% (w/v), but it is not limited thereto.

In the aforementioned second agent, the content of the aforementioned enzyme inhibitory component may be about 0.001-0.05% (w/v), such as about 0.0015-0.05% (w/v), about 0.002-0.045% (w/v), about 0.003-0.04% (w/v), about 0.004-0.035% (w/v), about 0.005-0.03% (w/v), about 0.008-0.025% (w/v), about 0.01-0.02% (w/v), about 0.001% (w/v), about 0.005% (w/v), about 0.007% (w/v), about 0.008% (w/v), about 0.01% (w/v), about 0.015% (w/v), about 0.016% (w/v), about 0.02% (w/v), about 0.025% (w/v), about 0.03% (w/v), about 0.035% (w/v), about 0.04% (w/v), about 0.05% (w/v), but it is not limited thereto.

In the aforementioned second agent, the content of the aforementioned metabolic inhibitory component may be about 0.1-2% (w/v), such as about 0.15-2% (w/v), about 0.2-2% (w/v), about 0.3-2% (w/v), about 0.35-1.5% (w/v), about 0.4-1.2% (w/v), about 0.5-1% (w/v), about 0.0-0.8% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.2% (w/v), about 1.4% (w/v), about 1.6% (w/v), about 1.8% (w/v), about 2% (w/v), but it is not limited thereto.

In the aforementioned second agent, the content of the aforementioned anticoagulant component may be about 0.5-89% (w/v), such as about 0.5-7.5% (w/v), about 1.2-7% (w/v), about 1.5-6.8% (w/v), about 1.8-6.6% (w/v), about 2-6.5% (w/v), about 2.2-6.4% (w/v), about 2.4.2-6.3% (w/v), about 2.6-6.2% (w/v), about 2.8-6.1% (w/v), about 3-6% (w/v), about 3.2-5.8% (w/v), about 3.4-5.6% (w/v), about 3.6-5.4% (w/v), about 3.8-5.2% (w/v), about 4-5% (w/v), about 0.5% (w/v), about 0.6% (w/v), about 0.8% (w/v), about 1% (w/v), about 1.2% (w/v), about 1.5% (w/v), about 2% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), about 3% (w/v), about 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4% (w/v), about 4.1% (w/v), about 4.2% (w/v), about 4.3% (w/v), about 4.4% (w/v), about 4.5% (w/v), about 4.6% (w/v), about 4.7% (w/v), about 4.8% (w/v), about 4.9% (w/v), about 5% (w/v), about 5.1% (w/v), about 5.2% (w/v), about 5.3% (w/v), about 5.4% (w/v), about 5.5% (w/v), about 5.6% (w/v), about 5.7% (w/v), about 5.8% (w/v), about 5.9% (w/v), about 6% (w/v), about 6.2% (w/v), about 6.4% (w/v), about 6.6% (w/v), about 6.8% (w/v), about 7% (w/v), about 7.2% (w/v), about 7.5% (w/v), about 8% (w/v), but it is not limited thereto.

In one embodiment, in the foregoing second agent, the content of the foregoing antiseptic component may be about 1-3% (w/v), the content of the foregoing enzyme inhibitory component may be about 0.001-0.05% (w/v), the content of the foregoing metabolic inhibitor may be about 0.1-2% (w/v), and the content of the foregoing anticoagulant component may be about 0.5-8% (w/v).

In addition, in another embodiment, in the in the second agent mentioned above, the antiseptic component mentioned above may include allantoin-formaldehyde condensation product while the allantoin-formaldehyde condensation product may be diazolidinyl urea, the enzyme inhibitor mentioned above may include a first metal chelator while the first metal chelator may be aurintricarboxylic acid, the metabolic inhibitory component may be NaF, and the anticoagulant component mentioned above may include a second metal chelator while the second metal chelator may be ethylenediaminetetraacetic acid, and the solvent mentioned above may be water. Furthermore, in this embodiment, in the second agent mentioned above, the content of the antiseptic component mentioned above may be about 1-3% (w/v), the content of enzyme inhibitory component mentioned above may be about 0.001-0.05% (w/v), the content of the metabolic inhibitor mentioned above may be about 0.1-2% (w/v) and the content of the anticoagulant component mentioned above may be about 0.5-8% (w/v). In one specific embodiment of this embodiment, in the second agent mentioned above, the content of the antiseptic component mentioned above may be about 1-1.8% (w/v), the content of enzyme inhibitory component mentioned above may be about 0.016% (w/v), and the content of the metabolic inhibitor mentioned above may be about 0.3% (w/v), and the anticoagulant component mentioned above may be about 1.2-5.8% (w/v).

In one specific embodiment, the aforementioned second agent consists of the aforementioned antiseptic component, the aforementioned enzyme inhibitory component, the aforementioned metabolic inhibitory component, the aforementioned anticoagulant component and the aforementioned solvent whole the aforementioned antiseptic component is diazolidinyl urea, the aforementioned enzyme inhibitor is aurintricarboxylic acid, the aforementioned metabolic inhibitory component is NaF, the aforementioned anticoagulant component is ethylenediaminetetraacetic acid and the aforementioned solvent is water. Furthermore, in this specific embodiment, in the second agent, the content of the aforementioned antiseptic component may be about 1-1.8% (w/v), the content of the aforementioned enzyme inhibitory component may be about 0.016% (w/v), the content of the aforementioned metabolic inhibitor may be about 0.3% (w/v), and the content of the aforementioned anticoagulant component may be about 1.2-5.8% (w/v).

By directly mixing whole blood or plasma with the stabilizing agent of the present disclosure mentioned above, the foregoing effects, such as maintaining the stability of the quality of cell-free nucleic acid and exosomal nucleic acid in whole blood, maintaining the integrity of white blood cells, and reducing the occurrence of hemolysis can be achieved.

Whole blood or plasma and the above-mentioned stabilizing agent for cell-free nucleic acid and/or exosomal nucleic acid in blood of the present disclosure can be mixed at a volume ratio of about 1:0.05-0.5, for example, can be mixed at a volume ratio of about 1:0.1-0.4, about 1:0.15-0.3, about 1:0.2-0.25, about 1:0.05, about 1:0.1, about 1:0.15, about 1:0.2, about 1:0.25, about 1:0.3, about 1:0.35, about 1:0.4, about 1:0.45, about 1:0.5, but it is mot limited thereto.

Based on the foregoing, the present disclosure may further provide a collection kit for cell-free nucleic acid and/or exosomal nucleic acid in blood.

The collection kit for cell-free nucleic acid and/or exosomal nucleic acid in blood of the present disclosure mentioned above may include, but is not limited to, any stabilizing agent for cell-free nucleic acid and/or exosomal nucleic acid in blood of the present disclosure mentioned above and a first container for accommodating a blood sample.

The material and/or shape of the first container has no particular limitation, as long as it does not have a negative impact on the blood sample. For example, the first container may include a centrifuge tube, a vacuum blood collection tube, but it is not limited thereto.

In one embodiment, in the collection kit for cell-free nucleic acid and/or exosomal nucleic acid in blood of the present disclosure mentioned above, the stabilizing agent for cell-free nucleic acid and/or exosomal nucleic acid in blood of the present disclosure may be prefilled in the first container mentioned above, and when the blood sample is obtained, the blood sample is directly loaded into the first container to directly mix with the stabilizing agent for cell-free nucleic acid and/or exosomal nucleic acid in blood of the present disclosure. In this embodiment, the first container mentioned above may be a centrifuge tube or a vacuum blood collection tube, etc., but it is not limited thereto.

In another embodiment, in the collection kit for cell-free nucleic acid and/or exosomal nucleic acid in blood of the present disclosure mentioned above, the stabilizing agent for cell-free nucleic acid and/or exosomal nucleic acid in blood of the present disclosure may be loaded in a second container, and when the blood sample is obtained, the blood sample is directly loaded into the first container, and the stabilizing agent for cell-free nucleic acid and/or exosomal nucleic acid in blood is removed from the second container mentioned above and added to the first container mentioned above to mix with the blood sample. The material and/or shape of the second container also has no particular limitation, as long as it does not have a negative impact on the stabilizing agent for cell-free nucleic acid and/or exosomal nucleic acid in blood of the present disclosure. For example, the second container may include a centrifuge tube, an ampoule, a plastic bottle, a glass bottle, but it is not limited thereto. Moreover, in this embodiment, the first container mentioned above may be a centrifuge tube or a vacuum blood collection tube, etc., but it is not limited thereto.

Moreover, the collection kit for cell-free nucleic acid and/or exosomal nucleic acid in blood of the present disclosure mentioned above may further include, but is not limited to, a nucleic acid extraction agent. The aforementioned nucleic acid extraction agent is used to extract the nucleic acid in the plasma of the blood sample after the blood sample is mixed with the stabilizing agent for cell-free nucleic acid and/or exosomal nucleic acid in the blood of the present disclosure. The aforementioned nucleic acid extraction agent may include a DNA extraction agent and/or a RNA extraction agent, but it is not limited thereto.

In addition, the present disclosure can also provide a use of a composition for In vitro stabilizing cell-free nucleic acid and/or exosomal nucleic acid in blood.

The composition mentioned in the use of a composition for in vitro stabilizing cell-free nucleic acid and/or exosomal nucleic acid in blood of the present disclosure may be any composition for stabilizing cell-free nucleic acid and/or exosomal nucleic acid in blood of the present disclosure mentioned above.

### EXAMPLES

### A. Methods

### 1. Preparation of Stock Solutions

### 1-1. Preparation of Stock Solution of 60% (w/v; g/104 mL) Diazolidinyl Urea (DU)

To 6 g of diazolidinyl urea powder, water was added to total volume to 10 mL to complete the preparation of a stock solution of 60% (w/v) diazolidinyl urea.

### 1-2. Preparation of Stock Solution of 0.1% (w/v) Aurintricarboxylic Acid (ATA)

To 0.01 g of aurintricarboxylic acid powder, water was added to total volume to 10 mL, and then filtered by a membrane filter with 0.22 μm pore size to complete the preparation of a stock solution of 0.1% (w/v) aurintricarboxylic acid.

### 1-3. Preparation of Stock Solution of 2% (w/v) NaF

To 0.2 g of NaF powder, water was added to total volume to 10 mL to complete the preparation of a stock solution of 0.2% (w/v) NaF.

### 1-4. Preparation of Stock Solution of 40% (w/v) Dipotassium Ethylenediaminetetraacetate (K₂EDTA)

To 4 g of dipotassium ethylenediaminetetraacetate powder, water was added to total volume to 10 mL to complete the preparation of a stock solution of 40% (w/v) dipotassium ethylenediaminetetraacetate.

### 2. Blood Processing

### 2-1. Mixing of Whole Blood and Stabilizing Agent

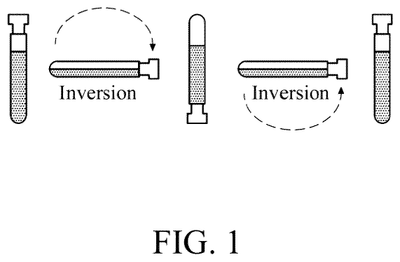

Whole blood and a stabilizing agent were mixed at a volume ratio of 1:0.15 in a test tube (10 mL whole blood+1.5 mL stabilizing agent or 2 mL whole blood+300 μL stabilizing agent) (commercially available products are based on the manufacturer's recommended ratio). The test tube was repeatedly and gently inverted up and down in the manner shown in FIG. 1 to mix the whole blood and the stabilizing agent evenly (avoiding shaking vigorously to prevent hemolysis).

### 2-2. Obtainment of Plasma

Blood having been mixed with a stabilizing agent was centrifuged at 2000×g for 15 minutes at 22° C. After that, the upper layer of liquid was taken to a new centrifuge tube and centrifuged again under the same condition to obtain the plasma in the upper layer.

### 3. Analysis of Cell-Free DNA in Blood

### 3-1. Extraction of Cell-Free DNA in Blood

Cell-free DNA in blood was extracted using the QIAamp Circulating Nucleic Acid Kit (QIAGEN) and referring to the manufacturer's manual for the following steps.
(1) 100 μL QIAGEN Proteinase K was pipetted into a 50 ml centrifuge tube.
(2) 1 mL of blood was added to the 50 mL centrifuge tube mentioned above.
(3) 0.8 mL Buffer ACL (containing 1.0 μg carrier RNA) was added into the 50 mL centrifuge tube mentioned above, and then the lid of the centrifuge tube was closed and the centrifuge tube was vortexed for mixing for 30 seconds.
(4) After mixing was complete, the centrifuge tube mentioned above was placed in a 60° C. oven and let stand for 30 minutes.
(5) After that, 1.8 mL Buffer ACB was added into the 50 mL centrifuge tube mentioned above, and then the lid of the centrifuge tube was closed and the centrifuge tube was vortexed for mixing for 15-30 seconds.
(6) The 50 mL centrifuge tube mentioned above was placed on ice for 5 minutes.
(7) QIAamp Mini column and collection tube were taken out, and the QIAamp Mini column was installed in the collection tube.
(8) 750 μL of lysate-buffer mixture solution was taken from the 50 mL centrifuge tube mentioned above and added to the QIAamp Mini column mentioned above.
(9) The QIAamp Mini column mentioned above was centrifuged at 1300 rpm for 1 minute.
(10) After centrifugation, the QIAamp Mini column was removed from the collection tube, and the liquid in the collection tube was discarded. Next, the QIAamp Mini column was installed back in the collection tube.
(11) Steps (8) to (10) were repeated until all the lysate-buffer mixture solution in the 50 mL centrifuge tube is added to the QIAamp Mini column and centrifugation was completed.
(12) 604 μL of Buffer ACW1 was added to the QIAamp Mini column, and then the QIAamp Mini column was centrifuged at 1300 rpm for 1 minute. After centrifugation, the QIAamp Mini column was removed from the collection tube, and the liquid in the collection tube was discarded. Next, the QIAamp Mini column was installed back in the collection tube.
(13) 750 μL of Buffer ACW2 was added to the QIAamp Mini column, and then the QIAamp Mini column was centrifuged at 1300 rpm for 1 minute. After centrifugation, the QIAamp Mini column was removed from the collection tube, and the liquid in the collection tube was discarded. Then, the QIAamp Mini column was installed back in the collection tube.
(14) 750 μL of ethanol (96-100%) was added to the QIAamp Mini column, and then the QIAamp Mini column was centrifuged at 1300 rpm for 1 minute. After centrifugation, the QIAamp Mini column was removed from the collection tube, and the liquid in the collection tube was discarded. Next, the QIAamp Mini column was installed back in the collection tube.
(15) The QIAamp Mini column was centrifuged at 1400 rpm for 3 minutes to completely move the liquid remaining on the membrane of the column to the collection tube, and then the liquid in the collection tube was discarded.
(16) The QIAamp Mini column was placed into a new collection tube, the lid of the collection tube was opened, and then the QIAamp Mini column was placed in a 56° C. dry bath for 10 minutes to allow the membrane of the column to completely dry.
(17) The collection tube was removed and the QIAamp Mini column was placed into a new 1.5 mL microcentrifuge tube.
(11) 22 μL of RNase Free Water was carefully added to the membrane on the QIAamp Mini column. After that, the lid of the microcentrifuge tube was closed, and the microcentrifuge tube was let to stand at room temperature for 3 minutes.
(19) The QIAamp Mini column was centrifuged at 14.000 rpm for 1 minute to transfer the solution containing cfDNA to the microcentrifuge tube.
(20) The QIAamp Mini column was removed, and the lid of the microcentrifuge tube was closed. The solution containing cfDNA obtained was in the microcentrifuge tube mentioned above.
(21) The obtained cfDNA solution was frozen and stored at −80° C. for later use.

### 3-2. Real-Time Polymerase Chain Reaction (qPCR) for Cell-Free DNA in Blood

The cell-free DNA in blood sample to be tested of was subjected to a real-time polymerase chain reaction by ABI 7500 Fast detection system (Applied Biosystems) with the wild-type exon 2 gene as the detection target.

First, a reaction solution was prepared. The total volume of the reaction solution was 20 μL. The reaction solution contained a sample of cell-free DNA in the blood to be tested (2 μL), a primer and probe mixed solution (containing a pair of self-designed primers (concentration of each primer was 125 nM) and a FAM/BHQ1 labeled probe (concentration of the probe was 100 nM)) (1 μL), 2× KAPA TagMan Master Mix (10 μL), 50× KAPA RTase Mix (KAPA PROBE FAST Universal One-Step qRT-PCR Master Mix Kit) (0.4 μL) and RNase-free water (6.6 μL).

The sequences of the primers and the probe for the wild-type exon 2 gene are shown in the following:

Forward primer:
(SEQ ID NO. 1)
AGGCACGAGTAACAAGCTC
Reverse primer:
(SEQ ID NO. 2)
CCACCTCACAGTTATTGAACATC
Probe:
(SEQ ID NO. 3)
CACGCAGTTGGGCACTTTTGAAGA

Next, the reaction solution mentioned above was subjected to a real-time polymerase chain reaction. The conditions for the real-time polymerase chain reaction are shown in the following:

42° C. for 5 minutes, 95° C. for 3 minutes, 95° C. for 3 seconds, and then 60° C. for 30 seconds. 40 cycles are performed.

### 3-3. Capillary Electrophoresis for Cell-Tree DNA in Blood

Cell-free DNA in blood was subjected to capillary electrophoresis by the Agilent High Sensitivity DNA kit (Agilent) according to the manufacturer's instructions, and then the capillary electrophoresis results were analyzed by an Agilent 2100 bioanalyzer to determine the molecular weight of cell-free DNA in the blood. The steps of capillary electrophoresis are summarized as follows:

First, a mixture of gel and dye was prepared according to the manufacturer's instructions.

Next, 9 μL of the mixture of gel and dye was added to each well of the high-sensitivity DNA chip in the kit, and then 1 μL of the sample of cell-free DNA in the blood to be tested was added into each well. Each high-sensitivity DNA chip can analyze 11 samples at most.

After the samples were added, the electrophoresis was started.

### 4. Analysis of Exosome RNA (exoRNA)

### 4-1. Extraction of Exosome RNA

Exosome RNA was extracted using the exoRNeasy Midi/Maxi kit (QIAGEN) and referring to the manufacturer's manual for the following steps.
(1) Plasma was filtered through a membrane filter with 4 nm pore size, and then 1 mL of filtered plasma was added to a new 1.5 mL microcentrifuge tube.
(2) 1 mL of Buffer XBP was to the microcentrifuge tube mentioned above, and the tube was inverted 5 times to mix plasma and Buffer XBP.
(3) exoEasy Midi spin column and collection tube were taken out, and the exoEasy Midi spin column was installed in the collection tube.
(4) The plasma/buffer XBP mixture was to the exoEasy spin column, and then centrifuged at 500×g for 1 minute. After centrifugation, the exoEasy spin column was removed from the collection tube, and the liquid in the collection tube was discarded. Next, the exoEasy spin column was installed back in the collection tube.
(5) 3.5 mL of Buffer XWP was added to the exoEasy spin column and centrifuged at 5000×g for 5 minutes. After centrifugation, the exoEasy spin column was removed from the collection tube, and the liquid in the collection tube and the collection tube were discarded.
(6) The exoEasy spin column was installed back in a new collection tube.
(7) 700 μL QIAzol was added to the membrane of the exoEasy spin column, and then centrifuged at 5000×g for 5 minutes. After that, the liquid obtained from the centrifugation was transferred to a new 2 mL test tube.
(8) The 2 mL test tube mentioned above was shaken briefly, and then allowed to stand at room temperature for 5 minutes.
(9) 90 μL of chloroform was added to the 2 mL test tube mentioned above, and then the test tube lid was closed and the test tube was shaken vigorously for 15 seconds to thoroughly mix the liquid in the test tube.
(10) After that, the 2 mL test tube mentioned above was allowed stand at room temperature for 2-3 minutes.
(11) The 2 mL tube mentioned above was centrifuged at 12,000×g for 15 minutes at 4° C. After centrifugation, the sample was divided into 3 phases: an upper layer of colorless aqueous phase containing RNA; a thin white interface; and a lower layer of red organic phase.
(12) The upper layer of aqueous phase (approximately 200 μL) was transferred to a new microcentrifuge tube, and care should be taken to avoid absorbing the above-mentioned white interface and red organic phase. Afterwards, 2 times the volume (400 μL) of 100% ethanol was added to the above-mentioned microcentrifuge tube containing the upper layer of aqueous phase, and the aqueous phase and ethanol was mixed through pipetting by a pipetman several times to obtain a mixture solution. The mixture solution was immediately used for the next step without centrifugation.
(13) RNeasy MinElute spin column and new collection tube were taken out, and the RNeasy MinElute spin column was installed in the collection tube.
(14) After that, the above-mentioned mixture solution (600 μL, including any precipitate that may have formed) was transferred to the RNeasy MinElute spin column entirely.
(15) Next, the RNeasy MinElute spin column was centrifuge at 8000×g for 15 seconds at room temperature. After centrifugation, exoEasy spin column was removed from the collection tube, and the liquid in the collection tube was discarded. Then, the RNeasy MinElute spin column was installed back in the collection tube.
(16) 700 μL of Buffer RWT was added to the RNeasy MinElute Spin Column and centrifuged at 8000×g for 15 seconds. After centrifugation. RNeasy MinElute spin column was removed from the collection tube, and the liquid in the collection tube was discarded. Next, the RNeasy MinElute spin column was installed back in the collection tube.
(17) 500 μL of Buffer RPE was added to the RNeasy MinElute spin column and centrifuged at 8000×g for IS seconds. After centrifugation, RNeasy MinElute spin column was removed from the collection tube, and the liquid in the collection tube was discarded. Next, the RNeasy MinElute spin column was installed back in the collection tube.
(18) 500 μL of Buffer RPE was added to the RNeasy MinElute spin column and centrifuged at 8000×g for 15 seconds. After centrifugation, RNeasy MinElute spin column was removed from the collection tube, and both of the collection tube and the liquid therein were discarded.
(19) The RNeasy MinElute spin column was placed into a new 2 mL test tube, the lid of the RNeasy MinElute spin column was opened and the RNeasy MinElute spin column was centrifuged at full speed for 5 minutes to dry the membrane of the RNeasy MinElute spin column. Afterwards, both of the 2 mL test tube and the liquid therein were discarded.
(18) The RNeasy MinElute spin column was placed into a new 1.5 L microcentrifuge tube. Next, 14 μL of RNase-free water was added to the membrane of the RNeasy MinElute spin column, and then the lid of the RNeasy MinElute spin column was closed and the RNeasy MinElute spin column was allowed to stand at room temperature for 1 minute.
(19) The RNeasy MinElute spin column was centrifuged at full speed for 5 minutes to transfer the exoRNA-containing solution to the microcentrifuge tube.
(20) The RNeasy MinElute spin column was removed, and lid of the microcentrifuge mentioned above tube was closed. The obtained exoRNA-containing solution was in the microcentrifuge tube mentioned above.
(21) The obtained exoRNA solution was frozen and stored at −80° C. for later use.

### 4-2. Reverse Transcription Real-Time Polymerize Chain Reaction for Exosome RNA

### 4-2-1. Reverse Transcription Reaction

The exosome RNA sample to be tested was subjected to a reverse transcription reaction by the GeneAmp PCR System 9700 detection system (Applied Biosystems) with miR-21 or miR-1228 as the target.

First, a reverse transcription reaction mixture was prepared. The total volume of the reverse transcription reaction mixture was 3 μL. The reverse transcription reaction mixture contained 100 mM dNTPs (0.15 μL), 50 U/μL MultiScribe Reverse Transcriptase (1 μL), 10× RT Buffer (1.5 μL), 20 U/μL RNase inhibitor (0.19 μL) and Nuclease-free Water (0.16 μL).

Next, a reaction solution was prepared. The total volume of the reverse transcription reaction solution was 15 μL, the reverse transcription reaction solution contained the reverse transcription reaction mixture prepared above (3 μL), primers (for miR-21 (manufacturer: ThermoFisher; product name: hsa-miR-21-5p; product number: 4427975; Assay ID: 000397) or miR-1228 (manufacturer: ThermoFisher; product name: hsa-miR-1228-5p; product number: 4427975; Assay ID: 002763)) (3 μL), exosome RNA sample to be tasted (4 μL) and ddH₂O (5 μL).

After that, the reaction solution mentioned above was subjected to a reverse transcription reaction to obtain a cDNA containing solution. The conditions for the reverse transcription reaction are shown in the following:
16° C. for 30 minutes, 42° C. for 30 minutes, and 85° C. for 5 minutes, after that 4° C. for storing.

### 4-2-2. Real-Time Polymerase Chain Reaction

The cDNA obtained above was subjected to a real-time polymerase chain reaction by ABI 7500 Fast detection system (Applied Biosystems) with the wild-type exon 2 gene as the detection target.

First, a reaction solution for polymerase chain reaction was prepared. The total volume of the reaction solution for polymerase chain reaction was 20 μL. The reaction solution for polymerase chain reaction contained the cDNA solution obtained above (9 μL was taken from the IS μL cDNA solution obtained above), a probe (for miR-21 (manufacturer: ThermoFisher; product name: hsa-miR-21-5p; product number: 4427975; Assay ID: 000397) or miR-1228 (manufacturer: ThermoFisher; product name: hsa-miR-1228-5p; product number: 4427975; Assay ID: 002763)) (1 μL) and 2× KAPA TaqMan Master Mix (10 μL) (Brand KAPA Biosystems; Brand name KAPA PROBE FAST Universal One-Step qRT-PCR Master Mix (2X) Kit; Cat. KK4752).

Next, the reaction solution mentioned above was subjected to a real-time polymerase chain reaction. The conditions for the real-time polymerase chain reaction are shown in the following:
42° C. for 5 minutes, 95° C. for 3 minutes, 95° C. for 3 seconds, and then 60° C. for 30 seconds. 40 cycles are performed.

### B. Experiments

### Example 1

### Efficacy Test of Stabilizing Agents Containing Different Concentrations of Diazolidinyl Urea (DU)

### 1. Preparation of Respective Formulas

Stabilizing agents containing formulas of different concentrations of diazolidinyl urea (DU) was prepared with the stock solution of 60% (w/v) diazolidinyl urea, the stock solution of 0.1% (w/v) aurintricarboxylic acid (ATA) and the stock solution of 2% (w/v) NaF prepared by the “1. Preparation of stock solution” in “A. Methods” above, further according to Table 1 shown in the following.

TABLE 1

| | Preparation |
|---|---|
| Formula 1 | Diazolidinyl urea (60%)*5 μL + Aurintricarboxylic acid (0.1%)*50 μL + NaF |
| | (2%)*50 μL + H₂O*195 μL |
| Formula 2 | Diazolidinyl urea (60%)*10 μL + Aurintricarboxylic acid (0.1%)*50 μL + |
| | NaF (2%)*50 μL + H₂O*190 μL |
| Formula 3 | Diazolidinyl urea (60%)*15 μL + Aurintricarboxylic acid (0.1%)*50 μL + |
| | NaF (2%)*50 μL + H₂O*185 μL |
| Formula 4 | Diazolidinyl urea (60%)*20 μL + Aurintricarboxylic acid (0.1%)*50 μL + |
| | NaF (2%)*50 μL + H₂O*180 μL |
| Formula 5 | Diazolidinyl urea (60%)*25 μL + Aurintricarboxylic acid (0.1%)*50 μL + |
| | NaF (2%)*50 μL + H₂O*175 μL |
| Formula 6 | Diazolidinyl urea (60%)*30 μL + Aurintricarboxylic acid (0.1%)*50 μL + |
| | NaF (2%)*50 μL + H₂O 170 μL |
| Formula 7 | Diazolidinyl urea (60%)*35 μL + Aurintricarboxylic acid (0.1%)*50 μL + |
| | NaF (2%)*50 μL + H₂O 165 μL |
| Formula 8 | Diazolidinyl urea (60%)*40 μL + Aurintricarboxylic acid (0.1%)*50 μL + |
| | NaF (2%)*50 μL + H₂O 160 μL |
| Formula 9 | Diazolidinyl urea (60%)*45 μL + Aurintricarboxylic acid (0.1%)*50 μL + |
| | NaF (2%)*50 μL + H₂O 155 μL |

Moreover, Table 2 in the following shows the content of each ingredient in each formula.

TABLE 2

| | Diazolidinyl | Aurintricarboxylic | | |
|---|---|---|---|---|
| | urea | acid | NaF | Note/Situation of mixing |
| | (%) | (%) | (%) | blood and the formula |
| Formula | 1 | 0.016 | 0.3 | Coagulation occurred on the |
| 1 | | | | day blood was mixed with the |
| | | | | formula, and thus blood |
| | | | | collected through EDTA blood |
| | | | | collection tubes* was used |
| | | | | instead to mix with the |
| | | | | formula. |
| Formula | 2 | | | Coagulation occurred on the |
| 2 | | | | day blood was mixed with the |
| | | | | formula, and thus blood |
| | | | | collected through EDTA blood |
| | | | | collection tubes* was used |
| | | | | instead to mix with the |
| | | | | formula. |
| Formula | 3 | | | Coagulation occurred on the |
| 3 | | | | day blood was mixed with the |
| | | | | formula, and thus blood |
| | | | | collected through EDTA blood |
| | | | | collection tubes* was used |
| | | | | instead to mix with the |
| | | | | formula. |
| Formula | 4 | | | Blood coagulation occurred |
| 4 | | | | after 3 days, and this formula |
| | | | | was eliminated. |
| Formula | 5 | | | No addition of EDTA** |
| 5 | | | | |
| Formula | 6 | | | No addition of EDTA** |
| 6 | | | | |
| Formula | 7 | | | No addition of EDTA** |
| 7 | | | | |
| Formula | 8 | | | No addition of EDTA** |
| 8 | | | | |
| Formula | 9 | | | No addition of EDTA** |
| 9 | | | | |

*According to the manufacturer's label, the K₂EDTA concentration of the EDTA blood collection tubes is 18.0 mg/mL.
**The blood collection tube does not contain any EDTA, since a certain concentration of diazolidinyl urea has an anticoagulant effect (about 5% or more of diazolidinyl urea has an anticoagulant effect).

### 2. Preservation of Whole Blood Samples

Based on the method described in “2-1. Mixing of whole blood and stabilizing agent” of “A. Methods” above, whole blood samples were mixed with stabilizing agents containing different formulas in Table 2 to form test samples. In addition, samples obtained from whole blood collected through commercial blood collection tubes (Streck) (Brand: Streck; Product name: Cell-Free DNA BCT; Catalog No. 218997), samples obtained from whole blood collected through commercial blood collection tubes (Qiagen) (Brand: Qiagen; Product name: PAXgene Blood DNA Tubes; Cat. No. 761115), and samples obtained from whole blood collected through commercial EDTA blood collection tubes (BD Vacutainer K2E (EDTA); Cat. No. 367525) were used as the samples of the control groups. Table 3 shows description of each sample.

TABLE 3

| Sample | Description of sample |
|---|---|
| Sample 1 | Mixture obtained by mixing a whole blood sample with Formula 1 |
| Sample 2 | Mixture obtained by mixing a whole blood sample with Formula 2 |
| Sample 3 | Mixture obtained by mixing a whole blood sample with Formula 3 |
| Sample 4 | Mixture obtained by mixing a whole blood sample with Formula 4 |
| Sample 5 | Mixture obtained by mixing a whole blood sample with Formula 5 |
| Sample 6 | Mixture obtained by mixing a whole blood sample with Formula 6 |
| Sample 7 | Mixture obtained by mixing a whole blood sample with Formula 7 |
| Sample 8 | Mixture obtained by mixing a whole blood sample with Formula 8 |
| Sample 9 | Mixture obtained by mixing a whole blood sample with Formula 9 |
| Qiagen | Whole blood was collected through a commercially available blood collection |
| | tube (Qiagen) according to the manufacturer's recommended manner, and the |
| | whole blood was stored directly therein. |
| Streck | Whole blood was collected through a commercially available blood collection |
| | tube (Streck) according to the manufacturer's recommended manner, and the |
| | whole blood was stored directly therein. |
| EDTA | Whole blood was collected through a commercially available EDTA blood |
| | collection tube* according to the manufacturer's recommended method, and |
| | the whole blood was stored directly therein. |

*According to the manufacturer's label, the K₂EDTA concentration of the EDTA blood collection tubes is 18.0 mg/mL.

After mixing, each sample was stored at room temperature for 7 days. In addition, each sample was analyzed on Day 7 after mixing.

### 3. Hemolysis Observation

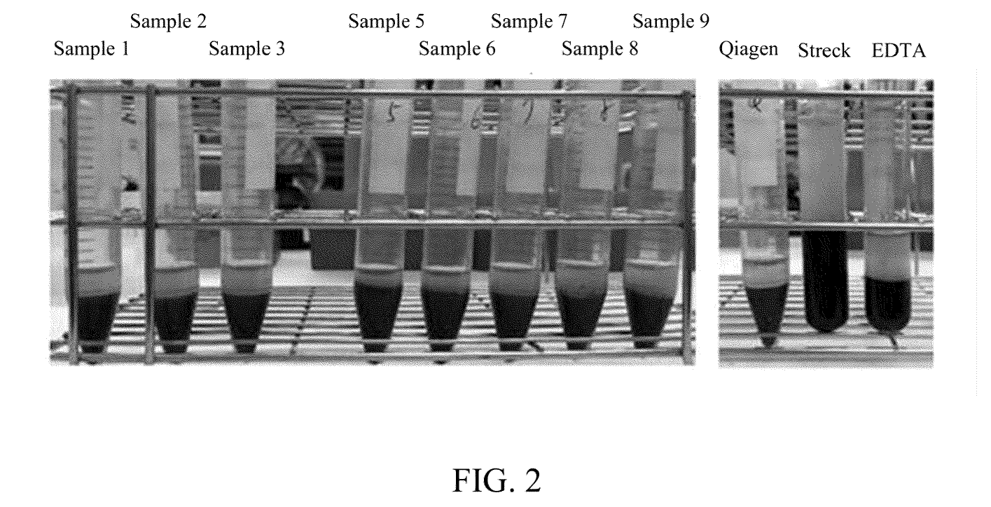

On Day 7 after mixing, the appearance of each test sample and the Qiagen control sample, the Streck control sample, and the EDTA control sample (whole blood was collected solely through an EDTA blood collection tube) (whole blood was collected solely through an EDTA blood collection tube) was observed. The results are shown in FIG. 2.

Based on FIG. 2, it should be understood that there is no significant difference in appearance between the test samples with different stabilizing agent formulas and the three control samples, and no obvious hemolysis occurred in any of them.

### 4. Analysis of Cell-Free DNA in Blood

### 4-1. Obtaining Plasma Cell-Free DNA

The plasma was obtained according to the method described in “2.2. Obtainment of plasma” in “A. Method:” above from the samples on Day 7 after mixing, and the plasma cell-free DNA was obtained according to the method described in “3-1. Extraction of cell-free DNA in blood” above from the obtained plasma.

### 4-2. Real-Time Polymerase Chain Reaction

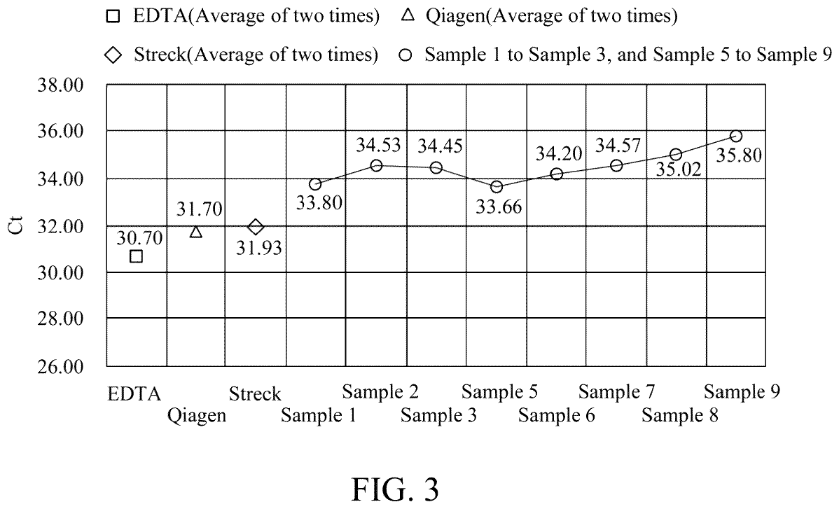

The plasma cell-free DNA obtained above was subjected to a real-time polymerase chain reaction according to the method described in “3-3. Real-time polymerase chain reaction (qPCR) for cell-free DNA in blood” in “A. Methods” above. The results are shown in FIG. 3.

Based on FIG. 3, it should be understood that Sample 1 to Sample 6 (in which the stabilizing agents contain 1-6% diazolidinyl urea) have better performance for Ct value. The Qiagen control sample, the Streck control sample and the EDTA control sample (whole blood was collected solely through the EDTA blood collection tubes) (whole blood was collected solely through the EDTA blood collection tubes) have lower Ct values due to the occurrence of cell decomposition, which resulted in a higher overall amount of nucleic acids.

### 4-3 Capillary Electrophoresis

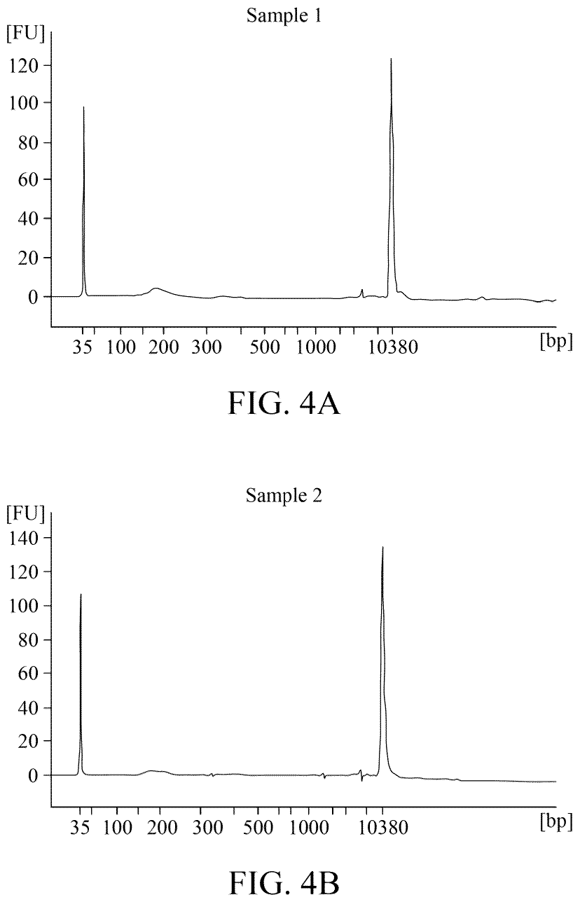

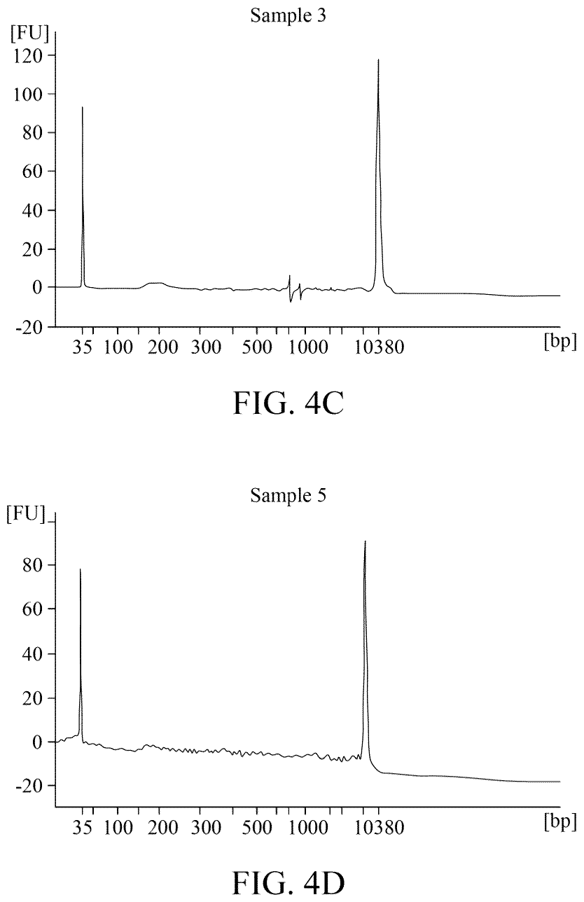

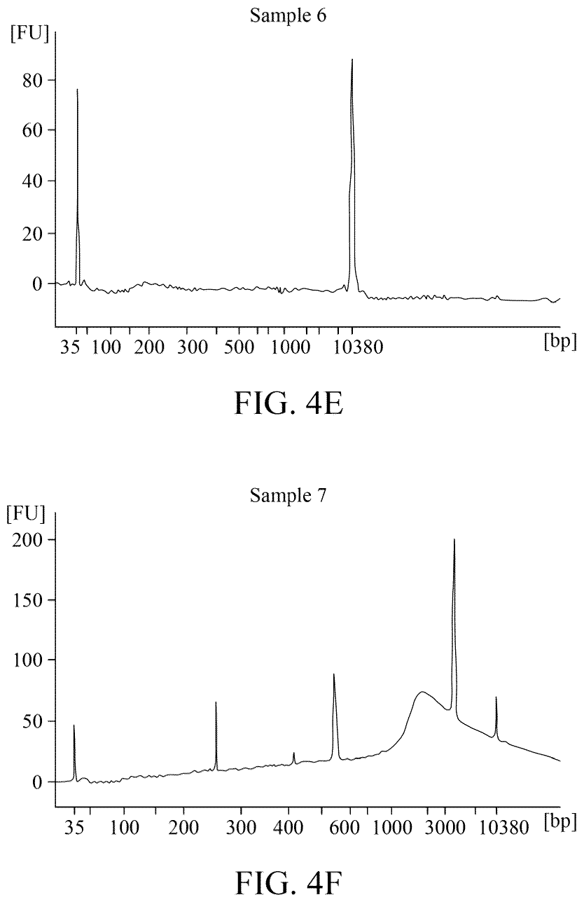

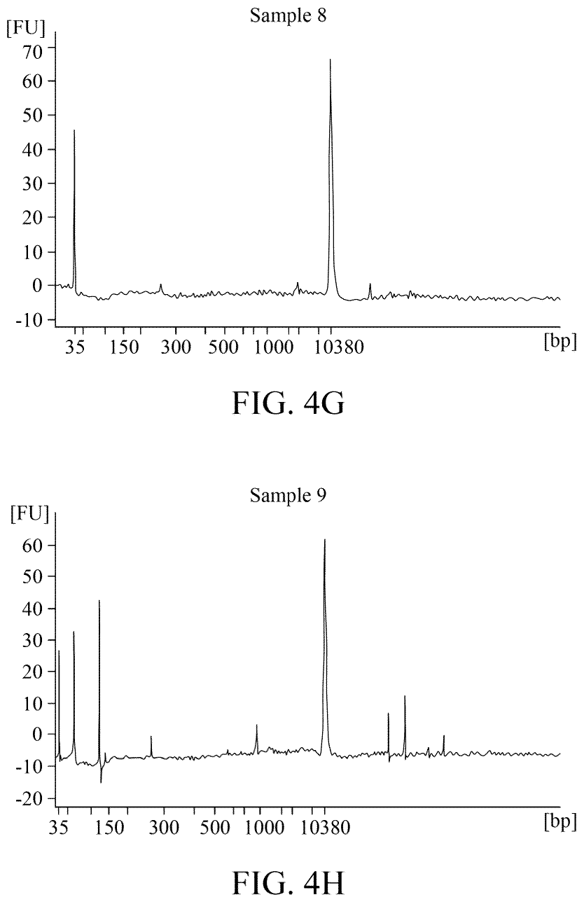

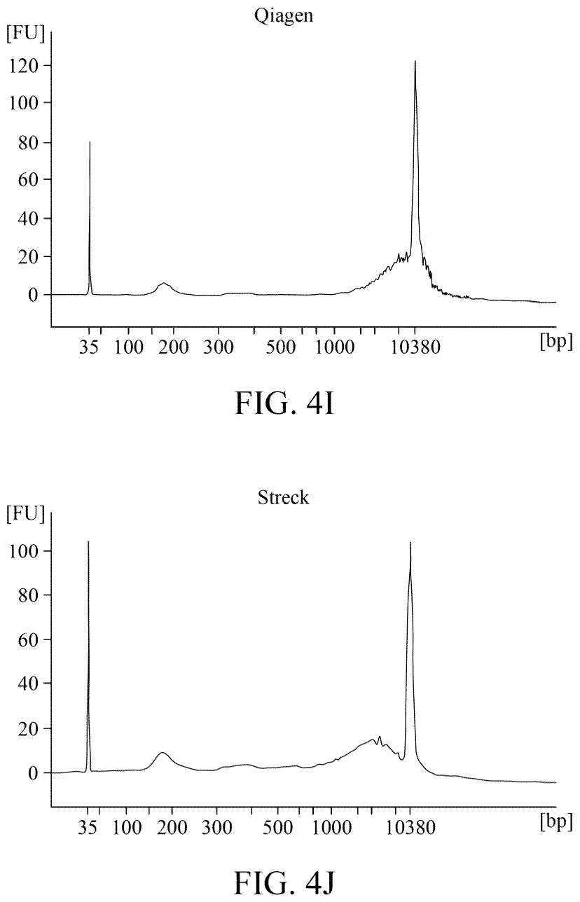

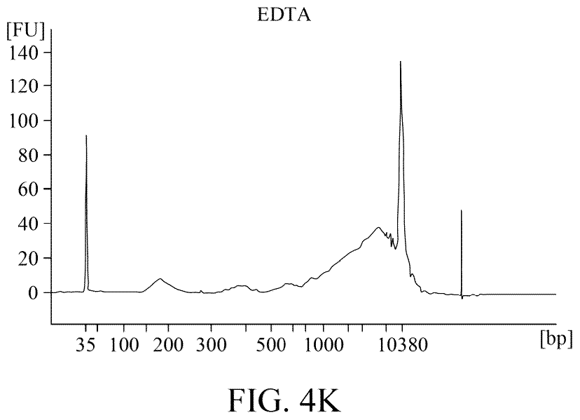

The plasma cell-free DNA obtained above was subjected to capillary electrophoresis according to the method described in “3-2. Capillary electrophoresis for cell-free DNA in blood” in “A. Methods” above. The results are shown in FIGS. 4A to 4K.

Based on FIGS. 4A to 4K it should be understood that Sample 1 (in which the stabilizing agent contains 1% diazolidinyl urea) shows the best cfDNA preservation effect (with an obvious peak at 150-200 bp, which is the size of cell-free nucleic acid), and no cell decomposition occurs (600-10380 bp interval is flat, indicating that there are no nucleic acid fragments with other lengths produced due to cell decomposition) (FIG. 4A). The Qiagen control sample (FIG. 4I), the Streck control sample (FIG. 4J) and the EDTA control sample (whole blood was collected solely through the EDTA blood collection tubes) (FIG. 4K) show the phenomenon of nucleic acid decomposition (600-10380 bp interval bulge). These results are consistent with the previous results of real-time polymerase chain reaction.

### 5. Conclusion

All test samples show no obvious hemolysis. In terms of capillary electrophoresis, Sample 1 (in which the stabilizing agent contains 1% diazolidinyl urea) performs best, while in terms of real-time polymerase chain reaction, Samples 1 to 6 (in which the stabilizing agents contains 1-6% diazolidinyl urea) are better.

### Example 2

### Efficacy Test of Stabilizing Agents Containing Different Concentrations of Diazolidinyl Urea (DU) (Whole Blood was Collected Through EDTA Blood Collection Tubes)

### 1. Preparation of Respective Formulas

Stabilizing agents containing formulas of different concentrations of diazolidinyl urea (DU) was prepared with the stock solution of 60% (w/v) diazolidinyl urea, the stock solution of 0.1% (w/v) aurintricarboxylic acid (ATA) and the stock solution of 2% (w/v) NaF prepared by the “1. Preparation of stock solution” in “A. Methods” above, further according to Table 4 shown in the following.

TABLE 4

| | Preparation |
|---|---|
| Formula | Diazolidinyl urea (60%)*1 μL + Aurintricarboxylic acid (0.1%)*50 μL + NaF |
| D1 | (2%)*50 μL + H₂O*199 μL |
| Formula | Diazolidinyl urea (60%)*2 μL + Aurintricarboxylic acid (0.1%)*50 μL + NaF |
| D2 | (2%)*50 μL + H₂O*198 μL |
| Formula | Diazolidinyl urea (60%)*3 μL + Aurintricarboxylic acid (0.1%)*50 μL + NaF |
| D3 | (2%)*50 μL + H₂O*197 μL |
| Formula | Diazolidinyl urea (60%)*4 μL + Aurintricarboxylic acid (0.1%)*50 μL + NaF |
| D4 | (2%)*50 μL + H₂O*196 μL |
| Formula | Diazolidinyl urea (60%)*5 μL + Aurintricarboxylic acid (0.1%)*50 μL + NaF |
| D5 | (2%)*50 μL + H₂O*195 μL |
| Formula | Diazolidinyl urea (60%)*6 μL + Aurintricarboxylic acid (0.1%)*50 μL + NaF |
| D6 | (2%)*50 μL + H₂O 194 μL |
| Formula | Diazolidinyl urea (60%)*7 μL + Aurintricarboxylic acid (0.1%)*50 μL + NaF |
| D7 | (2%)*50 μL + H₂O 193 μL |
| Formula | Diazolidinyl urea (60%)*8 μL + Aurintricarboxylic acid (0.1%)*50 μL + NaF |
| D8 | (2%)*50 μL + H₂O 192 μL |
| Formula | Diazolidinyl urea (60%)*9 μL + Aurintricarboxylic acid (0.1%)*50 μL + NaF |
| D9 | (2%)*50 μL + H₂O 191 μL |

Moreover, Table 5 in the following shows the content of each ingredient in each Formula.

TABLE 5

| | Diazolidinyl | Aurintricarboxylic | | |
|---|---|---|---|---|
| | urea | acid | NaF | Note/Situation of mixing |
| Formula | (%) | (%) | (%) | blood and the formula |
| Formula | 0.2 | 0.016 | 0.3 | Subsequently, whole blood |
| D1 | | | | will be collected through |
| Formula | 0.4 | | | commercially available |
| D2 | | | | EDTA blood collection |
| Formula | 0.6 | | | tubes* according to the |
| D3 | | | | manufacturer's recommended |
| Formula | 0.8 | | | method, and then 2 mL of the |
| D4 | | | | whole blood sample obtained |
| Formula | 1.0 | | | thereby will be mixed with |
| D5 | | | | the formula. |
| Formula | 1.2 | | | |
| D6 | | | | |
| Formula | 1.4 | | | |
| D7 | | | | |
| Formula | 1.6 | | | |
| D8 | | | | |
| Formula | 1.8 | | | |
| D9 | | | | |

*According to the manufacturer's label, the K2EDTA concentration of the EDTA blood collection tubes is 18.0 mg/mL
After calculation, and after the whole blood samples obtained through EDTA blood collection tubes are mixed with each respective formula, it is equivalent to a K2EDTA content of 1.2% in each of the formulas mentioned above.

### 2. Preservation of Whole Blood Samples

Based on the method described in “2-1. Mixing of whole blood and stabilizing agent” of “A. Methods” above, whole blood samples obtained through commercially available EDTA blood collection tubes (BD Vacutainer K2E (EDTA); Cat. No. 367525) were mixed with stabilizing agents containing different formulas in Table 5 to form test samples. In addition, the whole blood samples obtained through commercial blood collection tubes (Streck) (Brand: Streck; Product name: Cell-Free DNA BCT; Catalog No. 218997) and the whole blood samples obtained through commercial blood collection tubes (Qiagen) (Brand: Qiagen: Product name: PAXgene Blood DNA Tubes: Cat. No. 761115) were used as the samples of the control groups. Table 6 shows description of each sample.

TABLE 6

| Sample | Description of sample |
|---|---|
| Sample | Mixture obtained by collecting whole blood through a commercially available |
| D1 | EDTA blood collection tube* according to the manufacturer's recommended |
| | manner, and then mixing 2 mL of the whole blood sample thereby with |
| | Formula D1 |
| Sample | Mixture obtained by collecting whole blood through a commercially available |
| D2 | EDTA blood collection tube* according to the manufacturer's recommended |
| | manner, and then mixing 2 mL of the whole blood sample thereby with |
| | Formula D2 |
| Sample | Mixture obtained by collecting whole blood through a commercially available |
| D3 | EDTA blood collection tube* according to the manufacturer's recommended |
| | manner, and then mixing 2 mL of the whole blood sample thereby with |
| | Formula D3 |
| Sample | Mixture obtained by collecting whole blood through a commercially available |
| D4 | EDTA blood collection tube* according to the manufacturer's recommended |
| | manner, and then mixing 2 mL of the whole blood sample thereby with |
| | Formula D4 |
| Sample | Mixture obtained by collecting whole blood through a commercially available |
| D5 | EDTA blood collection tube* according to the manufacturer's recommended |
| | manner, and then mixing 2 mL of the whole blood sample thereby with |
| | Formula D5 |
| Sample | Mixture obtained by collecting whole blood through a commercially available |
| D6 | EDTA blood collection tube* according to the manufacturer's recommended |
| | manner, and then mixing 2 mL of the whole blood sample thereby with |
| | Formula D6 |
| Sample | Mixture obtained by collecting whole blood through a commercially available |
| D7 | EDTA blood collection tube* according to the manufacturer's recommended |
| | manner, and then mixing 2 mL of the whole blood sample thereby with |
| | Formula D7 |
| Sample | Mixture obtained by collecting whole blood through a commercially available |
| D8 | EDTA blood collection tube* according to the manufacturer's recommended |
| | manner, and then mixing 2 mL of the whole blood sample thereby with |
| | Formula D8 |
| Sample | Mixture obtained by collecting whole blood through a commercially available |
| D9 | EDTA blood collection tube* according to the manufacturer's recommended |
| | manner, and then mixing 2 mL of the whole blood sample thereby with |
| | Formula D9 |
| Qiagen | Whole blood was collected through a commercially available blood collection |
| | tube (Qiagen) according to the manufacturer's recommended manner, and the |
| | whole blood was stored directly therein. |
| Streck | Whole blood was collected through a commercially available blood collection |
| | tube (Streck) according to the manufacturer's recommended manner, and the |
| | whole blood was stored directly therein. |

*According to the manufacturer's label, the K₂EDTA concentration of the EDTA blood collection tubes is 18.0 mg/mL.

After mixing, each sample was stored at room temperature for 7 days. In addition, each sample was analyzed on Day 7 after mixing.

### 3. Hemolysis Observation

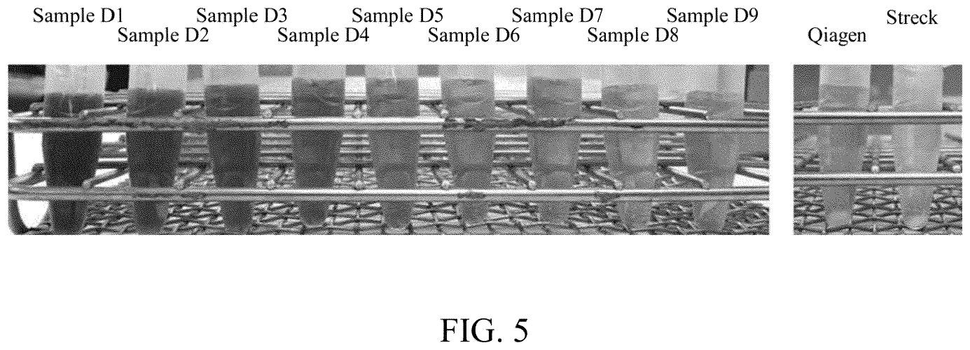

On Day 7 after mixing, the appearance of each test sample and the Qiagen control sample, the Streck control sample was observed. The results are shown in FIG. 5.

Based on FIG. 5, it should be understood that hemolysis occurs in the test samples containing formulas of low concentration diazolidinyl urea. As the concentration of diazolidinyl urea in the stabilizing agent formula increases, the hemolysis condition gradually improves. The hemolysis-inhibiting effect of stabilizing agent formulas with a diazolidinyl urea concentration increased to approximately 1% or above is comparable to that of commercially available blood collection tubes (Qiagen).

### 4. Analysis of Cell-Free DNA in Blood

### 4-1. Obtaining Plasma Cell-Free DNA

The plasma was obtained according to the method described in “2-2. Obtainment of plasma” in “A. Methods” above from the samples on Day 7 after mixing, and the plasma cell-free DNA was obtained according to the method described in “3-1. Extraction of cell-free DNA in blood” above from the obtained plasma

### 4-2. Real-Time Polymerase Chain Reaction

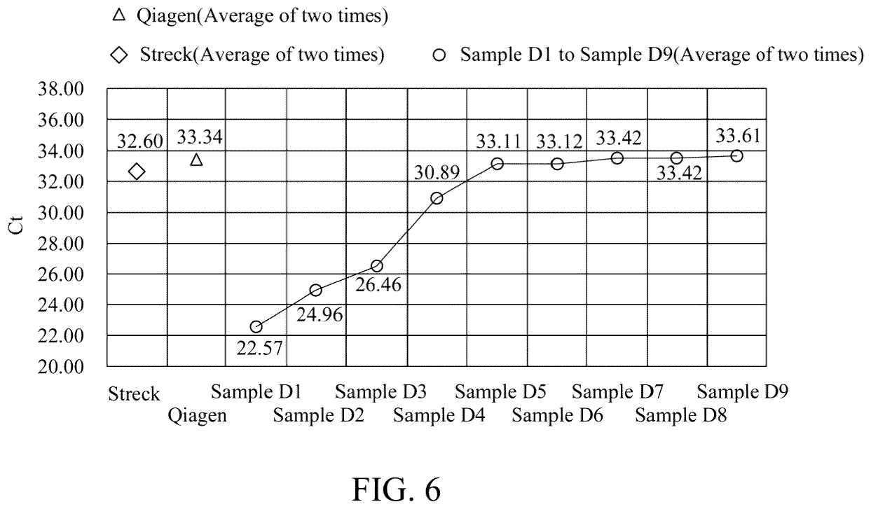

The plasma cell-free DNA obtained above was subjected to a real-time polymerase chain reaction according to the method described in “3-2. Real-time polymerase chain reaction (qPCR) for cell-free DNA in blood” in “A. Methods” above. The results are shown in FIG. 6.

Based on FIG. 6, it should be understood that the Ct values of Sample D1 to Sample D4 (in which the stabilizing agents contain 0.2-0.8% diazolidinyl urea) are relatively high, representing the presence of nucleic acid decomposition. As the concentration of diazolidinyl urea in the stabilizing agent formulas increases, the nucleic acid decomposition condition gradually improves. The Ct values of Sample D5 to Sample D9 (in which the stabilizing agent contain 1-1.8% diazolidinyl urea) are similar to those of the Qiagen control sample (Ct=33.34) and the Streck control sample (Ct=32.6), indicating that the efficiencies of inhibiting nucleic acid decomposition of the stabilizing agents therein are comparable to those of the commercially available products.

### 4-3 Capillary Electrophoresis

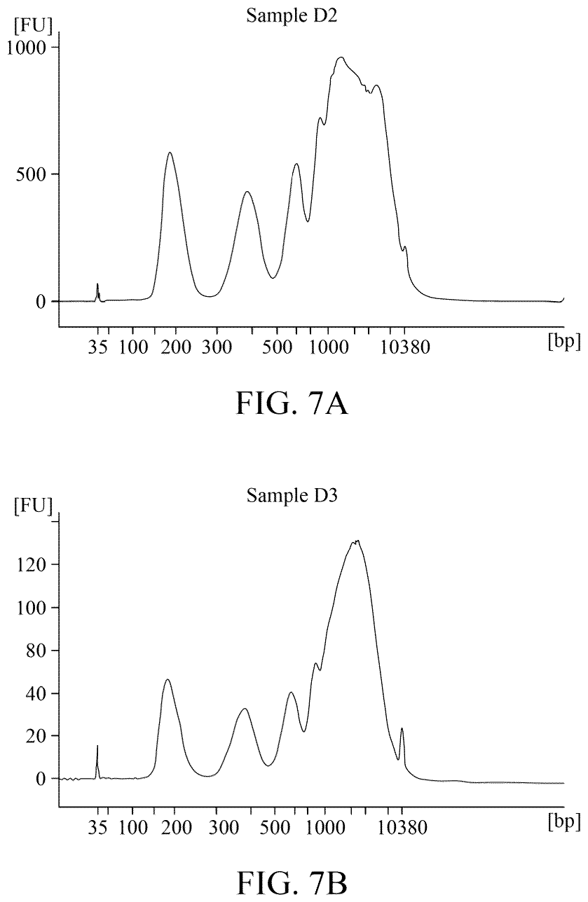

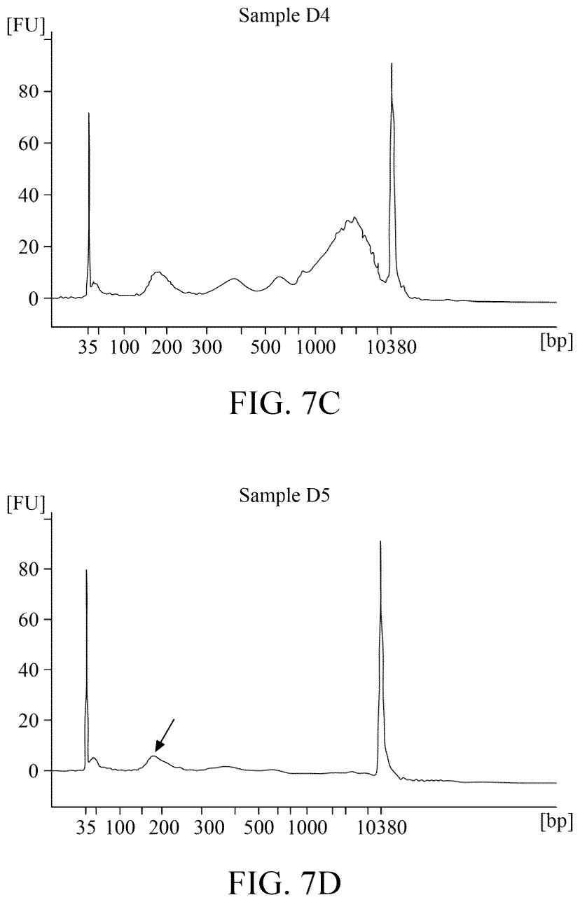

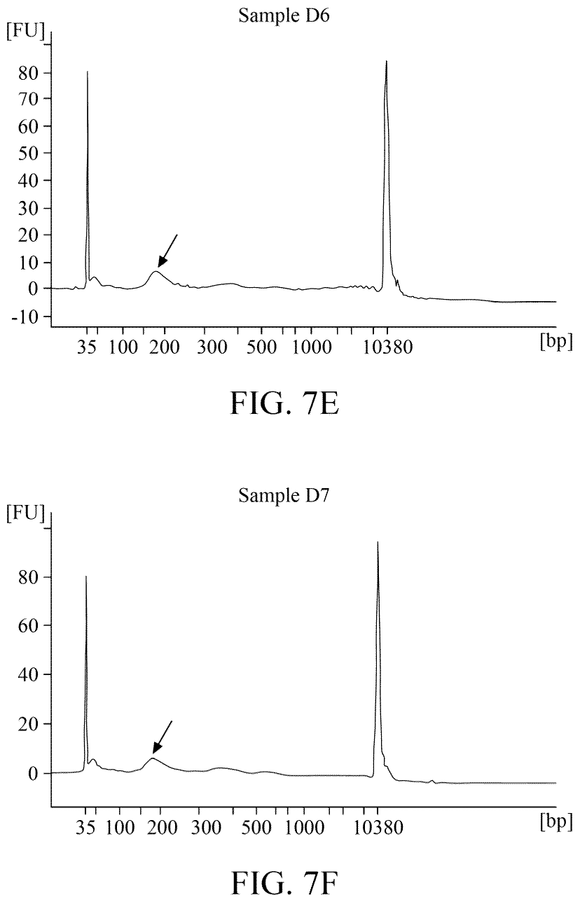

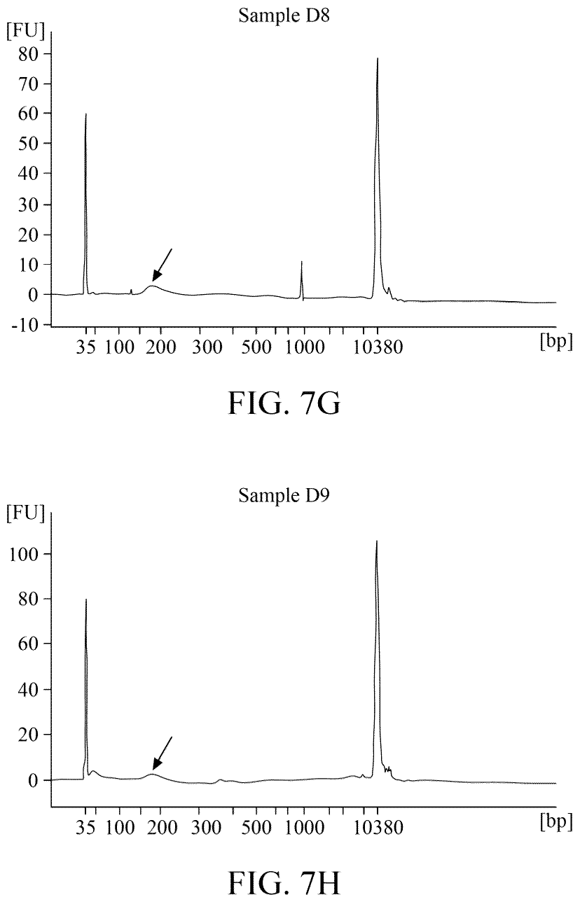

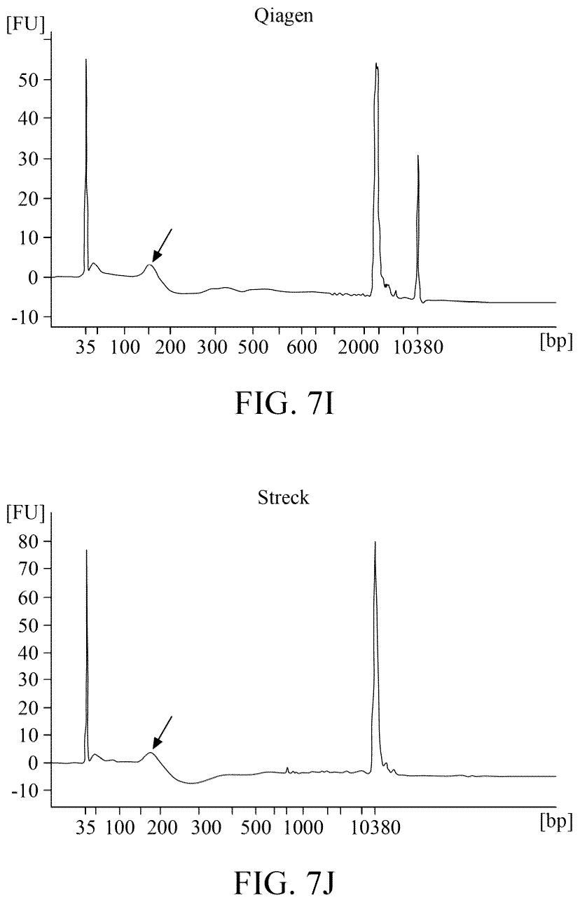

The plasma cell-free DNA obtained above was subjected to capillary electrophoresis according to the method described in “3-3. Capillary electrophoresis for cell-free DNA in blood” in “A. Methods” above (since the hemolysis of Sample D1 was particularly severe and its real-time polymerase chain reaction result showed obvious nucleic acid decomposition, capillary electrophoresis was not performed on it). The results are shown in FIGS. 7A to 7J.

Based on FIGS. 7A to 7J, it should be understood that Sample D2 to Sample D4 (in which the stabilizing agents contain 0.2-0.8% diazolidinyl urea) have nucleic acid fragments of different sizes (the results of capillary electrophoresis show multiple peaks at different positions), representing the presence of nucleic acid decomposition. Sample D5 to Sample D9 (in which the stabilizing agent contain 1-1.8% diazolidinyl urea) do not contain the different nucleic acid fragments mentioned above, and the results of capillary electrophoresis thereof show the peak of cfDNA (indicated by an arrow: 150-200 bp), indicating that the stabilizing agents therein are capable of effectively inhibiting the decomposition of nucleic acids and maintaining the integrity of cfDNA, and their effects are similar to those of commercially available blood collection tubes (Qiagen and Streck) (please referring to FIG. 7I and FIG. 7J).

### 5. Conclusion

According to the respective test results, it should be understood that stabilizing agent formulas D5 to D9 show better efficacy in all aspects compared to stabilizing agent formulas D1 to D4. That is to say, the stabilizing agents contain about 1% or more diazolidinyl urea, which have better efficacies of inhibiting hemolysis and stabilizing nucleic acids.

### Example 3

Efficacy test of stabilizing agents containing different concentrations of EDTA

### 1. Preparation of Various Formulas

Stabilizing agent formulas containing different concentrations of EDTA were prepared with the stock solution of 60% (w/v) diazolidinyl urea, the stock solution of 0.1% (w/v) aurintricarboxylic acid (ATA) and the stock solution of 2% (w/v) NaF prepared by the “1. Preparation of stock solution” in “A. Methods” above and a stock solution of 44% (w/v) dipotassium ethylenediaminetetraacetate (K₂EDTA), further according to Table 7 shown in the following.

TABLE 7

| | Preparation |
|---|---|
| Formula | Diazolidinyl urea (60%)*5 μL + Aurintricarboxylic acid (0.1%)*50 μL + NaF |
| E1 | (2%)*50 μL + 40% (w/v) K₂EDTA 19.5 μL + H₂O 175.5 μL |
| Formula | Diazolidinyl urea (60%)*5 μL + Aurintricarboxylic acid (0.1%)*50 μL + NaF |
| E2 | (2%)*50 μL + 40% (w/v) K₂EDTA 21 μL + H₂O 174 μL |
| Formula | Diazolidinyl urea (60%)*5 μL + Aurintricarboxylic acid (0.1%)*50 μL + NaF |
| E3 | (2%)*50 μL + 40% (w/v) K₂EDTA 22.5 μL + H₂O 172.5 μL |
| Formula | Diazolidinyl urea (60%)*5 μL + Aurintricarboxylic acid (0.1%)*50 μL + NaF |
| E4 | (2%)*50 μL + 40% (w/v) K₂EDTA 24 μL + H₂O 171 μL |
| Formula | Diazolidinyl urea (60%)*5 μL + Aurintricarboxylic acid (0.1%)*50 μL + NaF |
| E5 | (2%)*50 μL + 40% (w/v) K₂EDTA 25.5 μL + H₂O 169.5 μL |
| Formula | Diazolidinyl urea (60%)*5 μL + Aurintricarboxylic acid (0.1%)*50 μL + NaF |
| E6 | (2%)*50 μL + 40% (w/v) K₂EDTA 27 μL + H₂O 168 μL |
| Formula | Diazolidinyl urea (60%)*5 μL + Aurintricarboxylic acid (0.1%)*50 μL + NaF |
| E7 | (2%)*50 μL + 40% (w/v) K₂EDTA 85.5 μL + H₂O 109.2 μL |
| Formula | Diazolidinyl urea (60%)*5 μL + Aurintricarboxylic acid (0.1%)*50 μL + NaF |
| E8 | (2%)*50 μL + 40% (w/v) K₂EDTA 36 μL + H₂O 159 μL |
| Formula | Diazolidinyl urea (60%)*5 μL + Aurintricarboxylic acid (0.1%)*50 μL + NaF |
| E9 | (2%)*50 μL + 40% (w/v) K₂EDTA 43.5 μL + H₂O 151.5 μL |

Moreover, Table 8 in the following shows the content of each ingredient in each formula.

TABLE 8

| | Diazolidinyl | Aurintricarboxylic | | |
|---|---|---|---|---|
| | urea | acid | NaF | K₂EDTA |
| Formula | (%) | (%) | (%) | (%) |
| Formula 1 | 1 | 0.016 | 0.3 | 2.6 |
| Formula 2 | | | | 2.8 |
| Formula 3 | | | | 3 |
| Formula 4 | | | | 3.2 |
| Formula 5 | | | | 3.4 |
| Formula 6 | | | | 3.6 |
| Formula 7 | | | | 3.8 |
| Formula 8 | | | | 4.8 |
| Formula 9 | | | | 5.8 |

* According to the manufacturer's label, the K₂EDTA concentration of the EDTA blood collection tubes is 18.0 mg/mL.

### 2. Preservation of Whole Blood Samples

Based on the method described in “2-1. Mixing of whole blood and stabilizing agent” of “A. Methods” above, whole blood samples were mixed with stabilizing agents containing different formulas in Table 5 to form test samples. In addition, samples obtained from whole blood collected through commercial blood collection tubes (Streck) (Brand: Streck; Product name: Cell-Free DNA BCT; Catalog No. 218997), samples obtained from whole blood collected through commercial blood collection tubes (Qiagen) (Brand: Qiagen; Product name: PAXgene Blood DNA Tubes; Cat. No. 761115), and samples obtained from whole blood collected through commercial EDTA blood collection tubes (BD Vacutainer K2E(EDTA); Cat. No. 367525) were used as the samples of the control groups. Table 9 shows description of each sample.

TABLE 9

| Sample | Description of sample |
|---|---|
| Sample E1 | Mixture obtained by mixing a whole blood sample with Formula E1 |
| Sample E2 | Mixture obtained by mixing a whole blood sample with Formula E2 |
| Sample E3 | Mixture obtained by mixing a whole blood sample with Formula E3 |
| Sample E4 | Mixture obtained by mixing a whole blood sample with Formula E4 |
| Sample E5 | Mixture obtained by mixing a whole blood sample with Formula E5 |
| Sample E6 | Mixture obtained by mixing a whole blood sample with Formula E6 |
| Sample E7 | Mixture obtained by mixing a whole blood sample with Formula E7 |
| Sample E8 | Mixture obtained by mixing a whole blood sample with Formula E8 |
| Sample E9 | Mixture obtained by mixing a whole blood sample with Formula E9 |
| Qiagen | Whole blood was collected through a commercially available blood collection |
| | tube (Qiagen) according to the manufacturer's recommended manner, and the |
| | whole blood was stored directly therein. |
| Streck | Whole blood was collected through a commercially available blood collection |
| | tube (Streck) according to the manufacturer's recommended manner, and the |
| | whole blood was stored directly therein. |
| EDTA | Whole blood was collected through a commercially available EDTA blood |
| | collection tube* according to the manufacturer's recommended method, and |
| | the whole blood was stored directly therein. |

*According to the manufacturer's label, the K₂EDTA concentration of the EDTA blood collection tubes is 18.0 mg/mL.

After mixing, each sample was stored at room temperature for 11 days. In addition, each sample was analyzed on Day 11 after mixing.

### 3. Hemolysis Observation

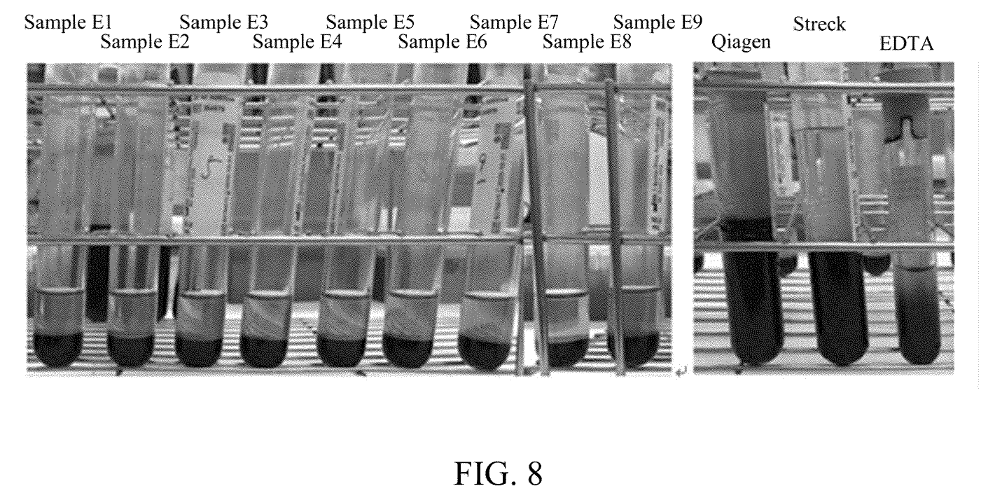

On Day 11 after mixing, the appearance of each test sample and the Qiagen control sample, the Streck control sample, and the EDTA control sample (whole blood was collected solely through an EDTA blood collection tube) was observed. The results are shown in FIG. 8.

Based on FIG. 8, it should be understood that after standing at room temperature for 11 days, there is no significant difference in the plasma volume of the blood samples mixed with respective formulas. In addition, except for Sample E8 and Sample E9, which show very slight hemolysis, there was no hemolysis in other test samples. In contrast, both the Qiagen control sample and the EDTA control sample (whole blood was collected solely through an EDTA blood collection tube) (whole blood was collected solely through an EDTA blood collection tube) show hemolysis.

### 4. Analysis of Cell-Free DNA in Blood

### 4-1. Obtaining Plasma Cell-Free DNA

The plasma was obtained according to the method described in “2-2. Obtainment of plasma” in “A. Methods” above from the samples on Day 7 after mixing, and the plasma cell-free DNA was obtained according to the method described in “3-1. Extraction of cell-free DNA in blood” above from the obtained plasma.

### 4-2 Real-Time Polymerase Chain Reaction

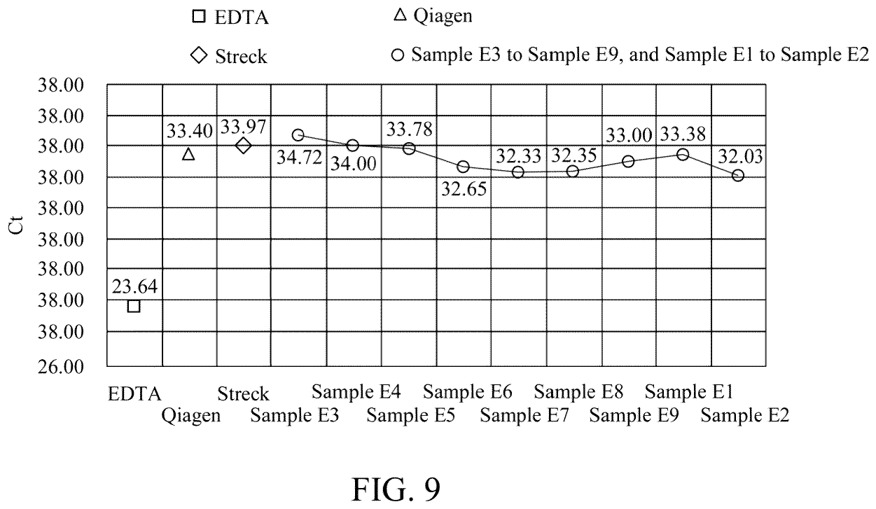

The plasma cell-free DNA obtained above was subjected to a real-time polymerase chain reaction according to the method described in “3-3. Real-time polymerase chain reaction (qPCR) for cell-free DNA in blood” in “A. Methods” above. The results are shown in FIG. 9.

FIG. 9 shows that, except for the slightly higher Ct value of Sample E3, there is no significant difference in the Ct values of the other test samples.

### 4-3. Capillary Electrophoresis

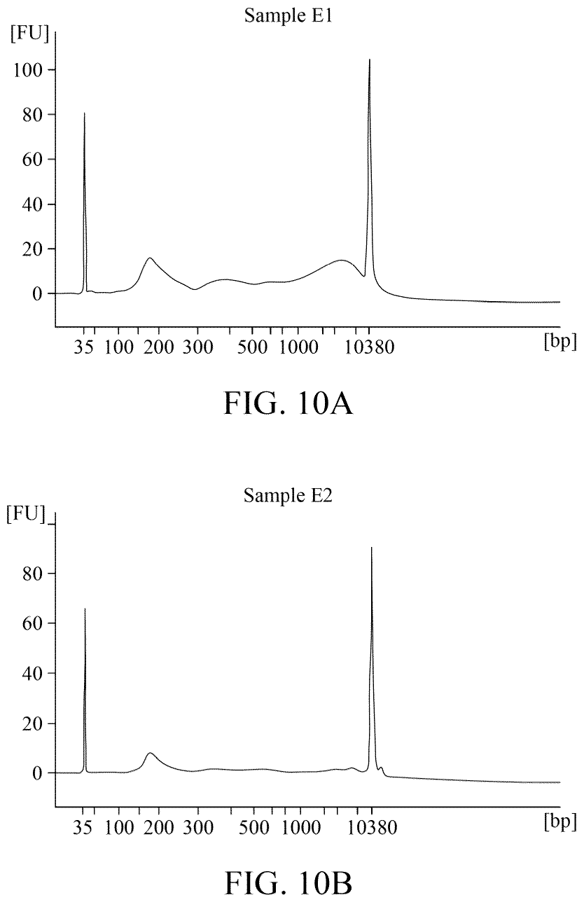

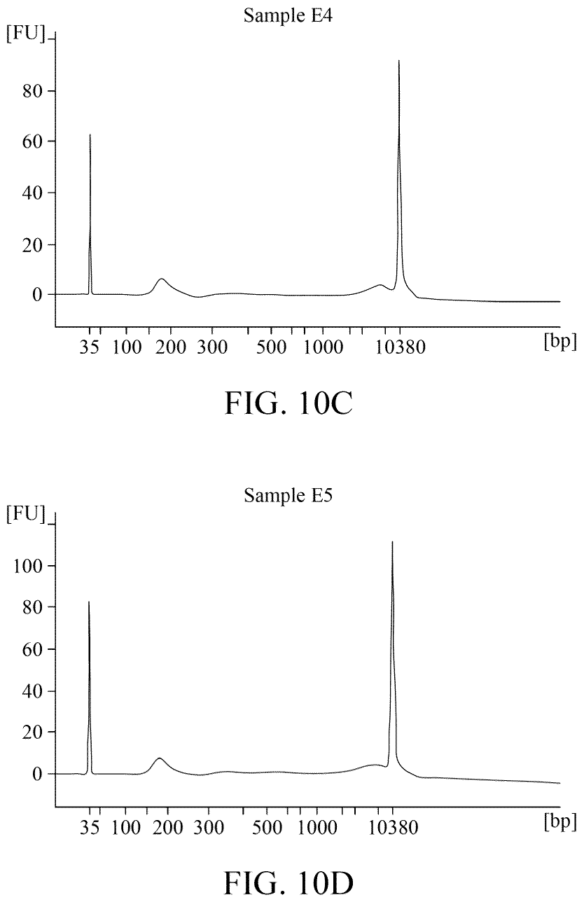

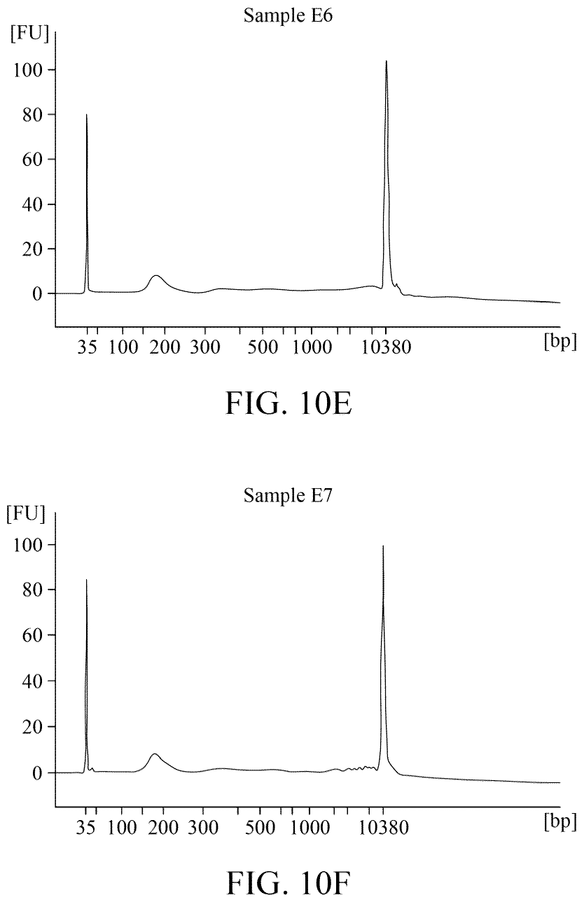

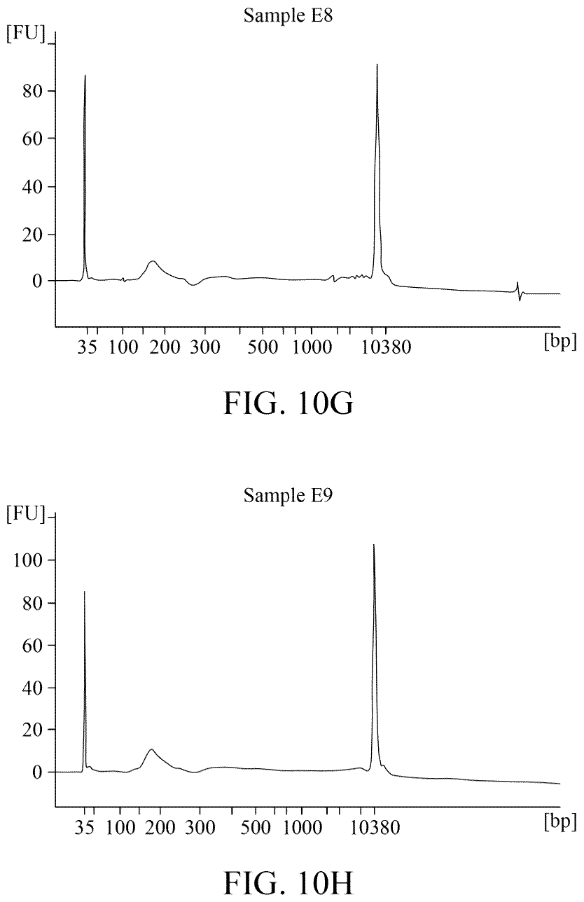

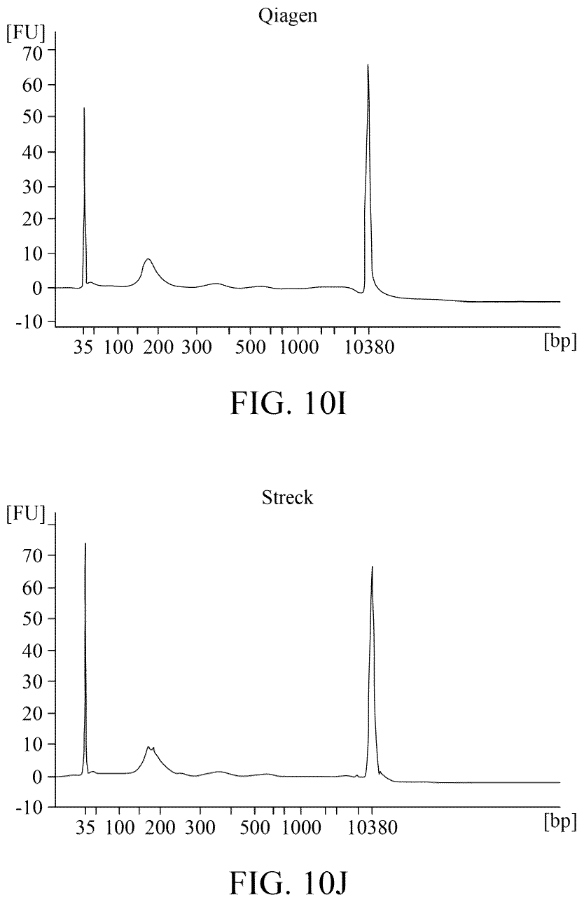

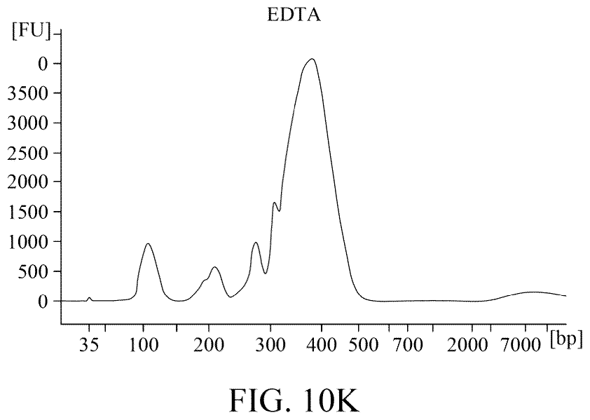

The plasma cell-free DNA obtained above was subjected to capillary electrophoresis according to the method described in “3-2. Capillary electrophoresis for cell-free DNA in blood” in “A. Methods” above. The results are shown in FIGS. 10A to 10K.

Based on FIGS. 10A to 10K, it should be understood that except for Sample E1 and Sample E2, which may have nucleic acid fragments with lengths other than 150-200 bp due to slight cell decomposition, other test samples all have the best cfDNA preserving effect (with obvious peaks at 150-200 bp), and no cell decomposition occurs (the 600-10380 bp interval is flat, indicating that there are no nucleic acid fragments with other lengths produced due to cell decomposition).

### 5. Conclusion

According to the respective test results mentioned above, it should be understood that the stabilizing agent formulas E4 to E9 show better efficacy in all aspects. Although there was slight hemolysis in Samples E8 and E9, that did not affect the results of the real-time polymerase chain reaction. In subsequent experiments, a formula with a near-median value of 3.8% EDTA was used.

### Example 4

### Long-Term Efficacy Test of Stabilizing Agents

### 1. Preparation of Formula

Stabilizing agents containing formulas of different concentrations of diazolidinyl urea (DU) was prepared with the stock solution of 60% (w/v) diazolidinyl urea, the stock solution of 0.1% (w/v) aurintricarboxylic acid (ATA) and the stock solution of 2% (w/v) NaF prepared by the “1. Preparation of stock solution” in “A. Methods” above and a stock solution of 40% (w/v) dipotassium ethylenediaminetetraacetate (K₂EDTA), further according to Table 10 shown in the following.

TABLE 10

Added volume (μL)

| | | |
|---|---|---|
| 60% (w/v) | 5 | 25 |
| Diazolidinyl urea | | |
| 0.1% (w/v) | 50 | 250 |
| Aurintricarboxylic acid | | |
| 2% (w/v) | 50 | 250 |
| NaF | | |
| K₂EDTA | 190 | 950 |
| H₂O | 5 | 25 |
| Total volume | 300 μL | 1500 μL |
| | 2 mL of whole | 10 mL of whole |
| | blood/tube | blood/tube |

Moreover, Table 11 in the following shows the content of each ingredient in the stabilizing agent.

TABLE 11

| | Diazolidinyl | Aurintricarboxylic | | |
|---|---|---|---|---|
| | urea | acid | NaF | K₂EDTA |
| | (%) | (%) | (%) | (%) |
| Formula | 1 | 0.016 | 0.3 | 3.8 |
| E7 | | | | |

### 2. Preservation of Whole Blood Samples

Based on the method described in “2-1. Mixing of whole blood and stabilizing agent” of “A. Methods” above, whole blood samples were mixed with the stabilising agent in Table 11 to form the test sample. In addition, samples obtained from whole blood collected through commercial blood collection tubes (Streck) (Brand: Streck; Product name: Cell-Free DNA BCT; Catalog No. 218997), and samples obtained from whole blood collected through commercial EDTA blood collection tubes (BD Vacutainer K2E(EDTA); Cat. No. 367525) were used as the samples of the control groups. Table 12 shows description of each sample. The experiments were performed in duplicate.

TABLE 12

| Sample | Description of sample |
|---|---|
| The present | Mixture obtained by mixing Formula E7 with a whole blood sample |
| disclosure | |
| Streck | Whole blood was collected through a commercially available blood |
| | collection tube (Streck) according to the manufacturer's recommended |
| | manner, and the whole blood was stored directly therein. |
| EDTA | Whole blood was collected through a commercially available EDTA |
| | blood collection tube* according to the manufacturer's recommended |
| | method, and the whole blood was stored directly therein. |

*According to the manufacturer's label, the K2EDTA concentration of the EDTA blood collection tubes is 18.0 mg/mL.

After mixing, each sample was stored at room temperature for 14 days. In addition, each sample was analyzed on Day 0, Day 4, Day 7 and Day 14 alter mixing.

### 3. Hemolysis Observation

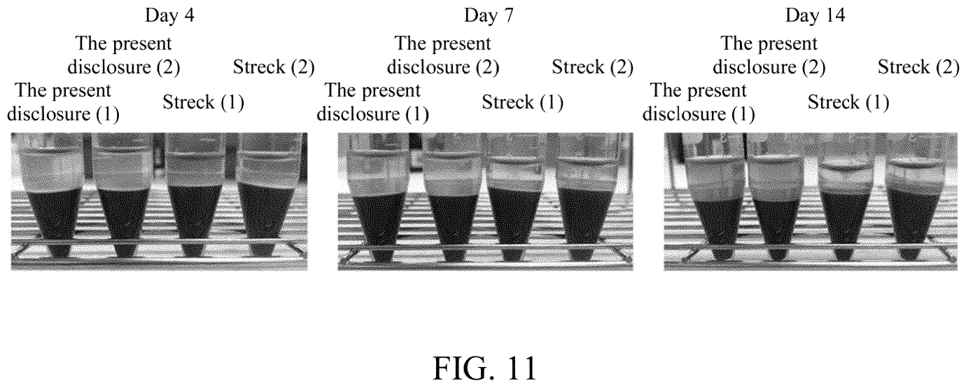

On Day 4, Day 7 and Day 14 after mixing, the appearance of the test sample of the present disclosure and the Streck control sample was observed. The results are shown in FIG. 11.

FIG. 11 shows that on the Day 4, there is no obvious difference in the appearance of the test sample of the present disclosure and the Streck control sample; on the Day 7, the Streck control sample has blood streaks in the plasma part, showing occurrence of hemolysis, while the plasma part of the test sample of the present disclosure is still clear, on Day 14, the test sample of the present disclosure shows slight turbidity in the plasma part, while the Streck control sample has blood streaks in the plasma pan, showing occurrence of hemolysis.

### 4. Analysis of Cell-Free DNA in Blood

### 4-1. Obtainment of Cell-Free DNA in Blood

The plasma was obtained according to the method described in “2-2. Obtainment of plasma” in “A. Methods” above from the samples on Day 0, Day 4, Day 7 and Day 14 after mixing, and the plasma cell-free DNA was obtained according to the method described in “3-1. Extraction of cell-free DNA in blood” above from the obtained plasma.

### 4-2. Real-Time Polymerase Chain Reaction

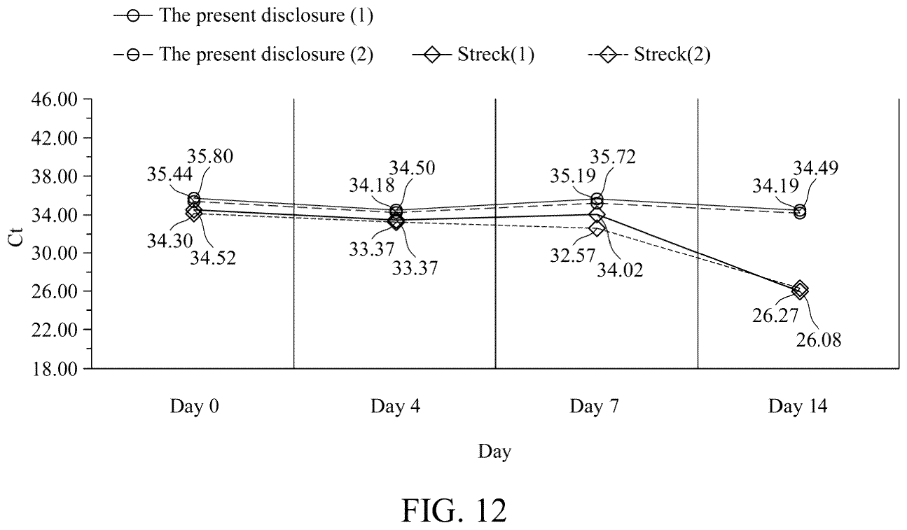

The plasma cell-free DNA obtained above was subjected to a real-time polymerase chain reaction according to the method described in “3-2. Real-time polymerase chain reaction (qPCR) for cell-free DNA in blood” in “A. Methods” above. Each sample was tested in duplicate. The results are shown in FIG. 12.

According to FIG. 12, it should be understood that on Day 0-7, the Ct values of the test sample of the present disclosure and the Streck control sample remain normal, and on the Day 14, the Ct value of the test sample of the present disclosure still has no significant change, but the Ct value of the Streck control sample shows a decrease, and this should be caused by the release of nucleic acid due to cell rupture in the Streck control sample.

### 4-3. Capillary Electrophoresis

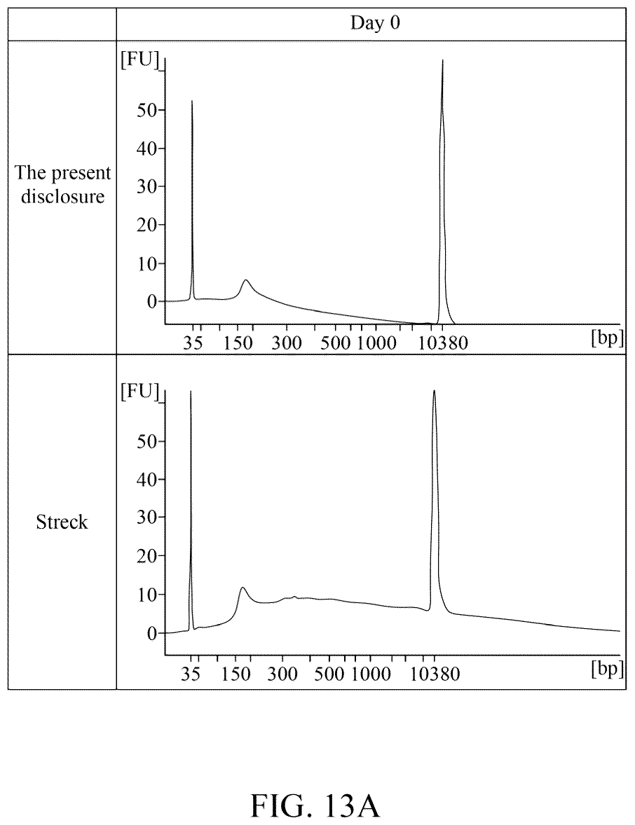

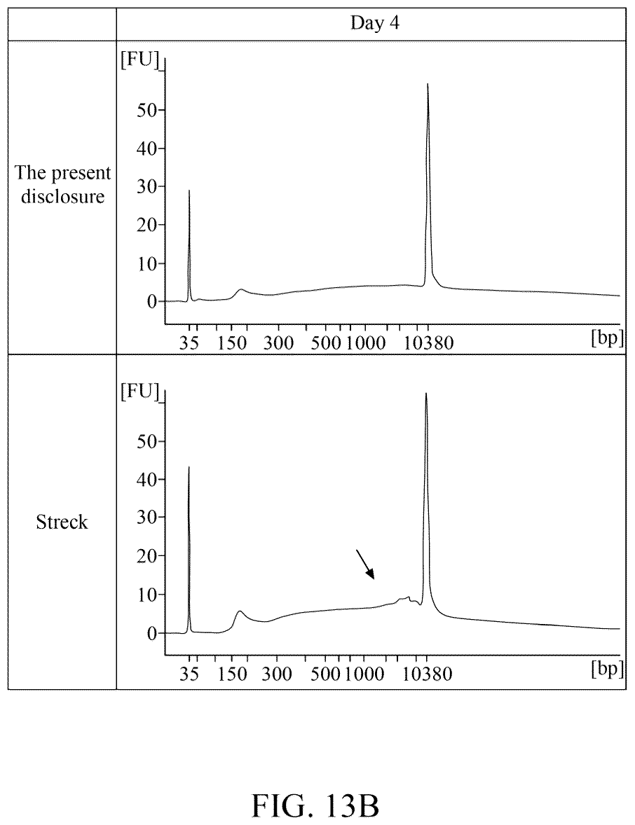

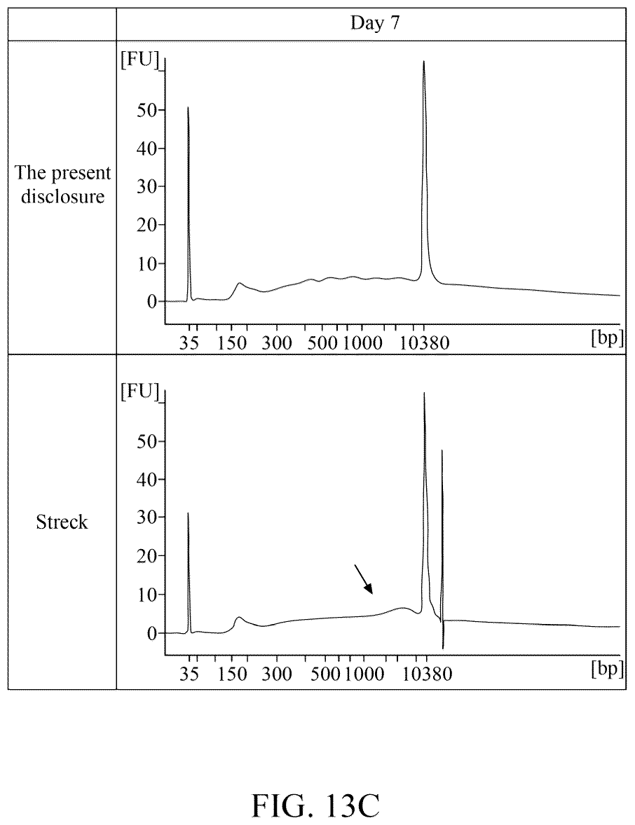

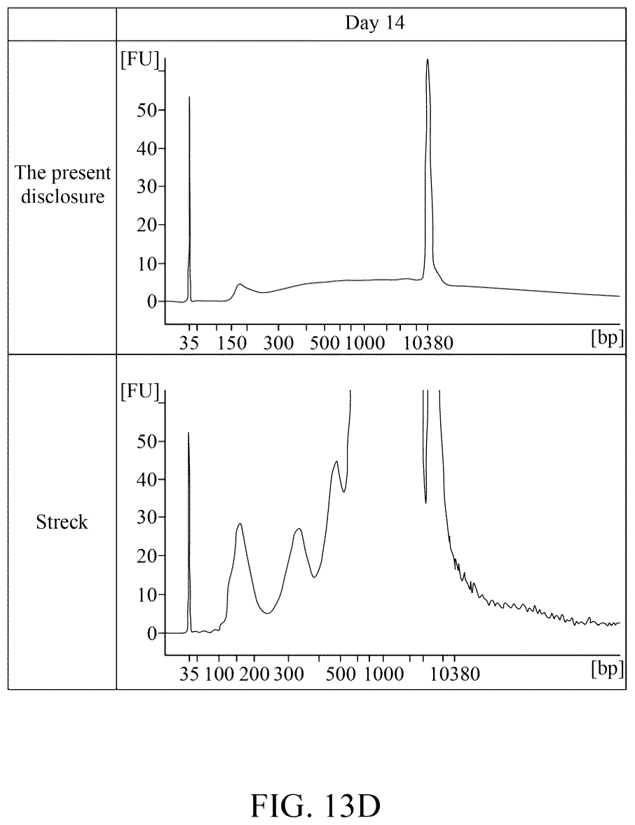

The plasma cell-free DNA obtained above was subjected to capillary electrophoresis according to the method described in “3-3. Capillary electrophoresis for cell-free DNA in blood” in “A. Methods” above. The results are shown in FIGS. 13A to 13D.

According to FIG. 13A, it should be understood that on Day 0, in the capillary electrophoresis patterns of the test sample of the present disclosure and the Streck control sample, a peak can be observed at a position approximately 100-200 bp of nucleic acid fragment size, which represents cell-free nucleic acid, while no peak is observed at the position of larger fragments, indicating that cell lysis in the blood is inhibited and no large amounts of nucleic acids are released due to cell rupture. FIGS. 13B to 13D show that on Days 4, 7 and 14, the test sample of the present disclosure still maintains the same situation as on Day 0. In contrast, although the Streck control sample maintains a peak value representing free DNA, it begins to have nucleic acid fragment peaks of different sizes, and it is estimated that this is due to cell lysis in the blood, resulting in a phenomenon of massive release of nucleic acids from cells (as indicated by the arrow), and this result is consistent with the results of real-time polymerase chain reaction.

### 5. Analysis of Exosomal Free RNA in Blood

### 5-1. Obtainment of Exosomal Free RNA in Blood

The plasma was obtained according to the method described in “2-2. Obtainment of plasma” in “A. Methods” above from the samples on Day 4 and Day 7 after mixing, and exosomal free RNA in blood was obtained according to the method described in “4-1. Extraction of exosome RNA of exosome RNA” above from the obtained plasma.

### 5-2. Reverse Transcription Real-Time Polymerase Chain Reaction

The plasma cell-free DNA obtained above was subjected to a real-time polymerase chain reaction according to the method described in “4-2. Reverse transcription real-time polymerase chain reaction for exosome RNA” in “A. Methods” above.

### 5-3. Calculation of 2^{-ΔΔCt} Value

2^{-ΔΔCt} values of miR-21 in real-time polymerase chain reaction for each of samples on Day 4 and Day 7 for the storage were calculated.

The calculation method is described as follow.

miR-1228 was used as the reference control for relative quantitative analysis of miR-21.

Ct values of miR-21 (hereinafter referred to as Ct_{miR-21}) and Ct values of miR-1228 (hereinafter referred to as Ct_{miR-1228}) presented in real-time polymerase chain reactions for samples on Day 0, Day 4 and Day 7 for the storage were determined.

The following describes the information will be received.

Ct_{miR-21} and Ct_{miR-1228} of the sample on Day 0 for the storage (hereinafter referred to as Ct_{miR-21[Day0]} and Ct_{miR-1228[Day0]}, respectively).

Ct_{miR-21} and Ct_{miR-1228} of the sample on Day 4 for the storage (hereinafter referred to as Ct_{miR-21[Day4]} and Ct_{miR-1228[Day4]}, respectively).

Ct_{miR-21} and Ct_{miR-1228} of the sample on Day 7 for the storage (hereinafter referred to as Ct_{miR-21[Day7]} and Ct_{miR-1228[Day7]}, respectively)

The ΔCt value of miR-21 is defined as the difference between the Ct value of miR-21 and the Ct value of miR-1228 (Ct_{miR-21}−Ct_{miR-1228}). The details are as follows:

ΔCt value of miR-21 of the sample on Day 0 for the storage (ΔCt_{Day0})=Ct_{miR-21[Day0]}−Ct_{miR-1228[Day0]}.

ΔCt value of miR-21 of the sample on Day 4 for the storage (ΔCt_{Day4})=Ct_{miR-21[Day4]}−Ct_{miR-1228[Day4]}.

ΔCt value of miR-21 of the sample on Day 7 for the storage (ΔCt_{Day7})=Ct_{miR-21[Day7]}−Ct_{miR-1228[Day7]}.

The ΔΔCt value of miR-21 of the sample on Day 4 for the storage is defined as the difference between ΔCt_{Day4} and ΔCt_{Day0}(ΔCt_{Day4}−ΔCt_{Day0}).

The ΔΔCt value of miR-21 of the sample on Day 7 for the storage is defined as the difference between ΔCt_{Day7} and ΔCt_{Day0}(ΔCt_{Day7}−ΔCt_{Day0}).

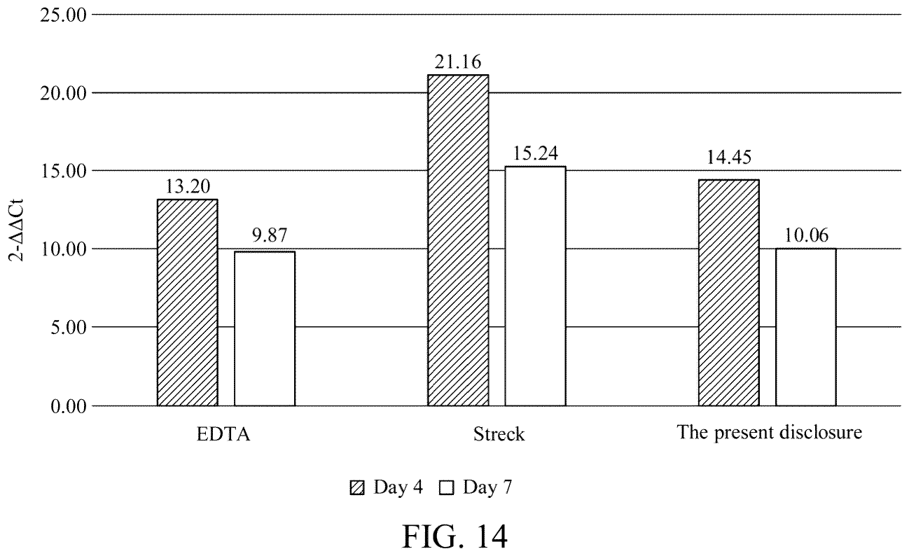

Finally, based on the information mentioned above, the 2^{-ΔΔCt} values of the real-time polymerase chain reaction for miR-21 in each of samples on Day 4 and Day 7 for the storage were calculated. The results are shown in FIG. 14.

The results show that the 2^{-ΔΔCt} values of miR-21 in the control samples Streck on Day 4 and Day 7 are significantly higher than that of the other two samples (test sample of the present disclosure and the EDTA control sample (whole blood was collected solely through EDTA blood collection tubes)).

Furthermore, exosomes derived from platelets containing high amounts of miR-21 is reported by literature (Thrombin-activated platelet-derived exosomes regulate endothelial cell expression of ICAM-1 via microRNA-223 during the thrombosis-inflammation response, Thromb Res., 2017, 154; 96-105), and thus it is speculated that the increase in miR-21 in the Streck control sample is caused by exosomes released after activation of platelets in plasma. In addition, literature reports that platelets will be activated during blood collection and release a large amount of exosomes, causing interference (Technical challenges of working with extracellular vesicles, Nanoscale, 2018, 10, 881-906), and a good blood collection tube must be able to suppress this phenomenon. According to literature reports, EDTA blood collection tubes can effectively inhibit the release of exosomes from platelets (Standardization of Blood Collection and Processing for the Diagnostic Use of Extracellular Vesicles. Curr. Pathobio. Rep., 2019, 1-8), and are suitable for exosome RNA analysis.

Furthermore, FIG. 14 shows that in addition to the EDTA control sample blood collection tubes (whole blood is collected solely through EDTA blood collection tube), the stabilizing agent of the present disclosure can also achieve the inhibitory effect.

### 6. Conclusion

Integrating the foregoing results, it should be understood that the stabilizing agents of the present disclosure are able to well maintain the cell-free DNA in the blood and are capable of maintaining the stability of the free nucleic acid in exosomes.

It will be apparent to those skilled in the art that various modifications and variations can be made to the disclosed embodiments. It is intended that the specification and examples be considered as exemplary only, with the true scope of the disclosure being indicated by the following claims and their equivalents.

What is claimed is:

1. A composition for stabilizing cell-free nucleic acid and/or exosomal nucleic acid in blood, which is selected from a group consisting of the following compositions (a) and (b):
(a) a first composition, which comprises:
(i) an antiseptic component, comprising:
an allantoin-formaldehyde condensation product;
(ii) an enzyme inhibitory component, comprising:
a first metal chelator; and
(iii) a metabolic inhibitory component, comprising:
NaF or sodium azide,
wherein a weight ratio of the antiseptic component, the enzyme inhibitory component and the metabolic inhibitory component is 1-3:0.001-0.05:0.005-0.08; and
(b) a second composition, which comprises:
(i) an antiseptic component, comprising:
an allantoin-formaldehyde condensation product;
(ii) an enzyme inhibitory component, comprising:
a first metal chelator;
(iii) a metabolic inhibitory component, comprising:
NaF or sodium azide; and
(iv) an anticoagulant component, comprising:
a second metal chelator,
wherein a weight ratio of the antiseptic component, the enzyme inhibitory component, the metabolic inhibitory component and the anticoagulant component is 1-3:0.001-0.05:0.1-2:0.5-8.

2. The composition for stabilizing cell-free nucleic acid and/or exosomal nucleic acid in blood as claimed in claim 1, wherein the cell-free nucleic acid in blood comprises cell-free DNA in blood, and the cell-free DNA in blood comprises circulating tumor DNA (ctDNA) and/or cell-free fetal DNA (cfDNA).

3. The composition for stabilizing cell-free nucleic acid and/or exosomal nucleic acid in blood as claimed in claim 1, wherein the exosomal nucleic acid comprises DNA or RNA in exosomes, and the RNA in the exosomal nucleic acid comprises messenger RNA (mRNA), microRNA (miRNA), long non-coding RNA (lncRNA) or competing endogenous RNA (ceRNA) in exosomes.

4. The composition for stabilizing cell-free nucleic acid and/or exosomal nucleic acid in blood as claimed in claim 1, wherein the allantoin-formaldehyde condensation product comprises at least one of diazolidinyl urea (DU) and imidazolidinyl urea (IDU), and the first metal chelator comprises aurintricarboxylic acid (ATA), and the second metal chelator comprises at least one of ethylenediaminetetraacetic acid (EDTA) and citrate (citrate).

5. The composition for stabilizing cell-free nucleic acid and/or exosomal nucleic acid in blood as claimed in claim 1, wherein the composition for stabilizing cell-free nucleic acid and/or exosomal nucleic acid in blood is the first composition, and wherein the allantoin-formaldehyde condensation product is diazolidinyl urea, the first metal chelator is aurintricarboxylic acid, and the metabolic inhibitory component is NaF.

6. The composition for stabilizing cell-free nucleic acid and/or exosomal nucleic acid in blood as claimed in claim 5, wherein a weight ratio of the antiseptic component, the enzyme inhibitory component and the metabolic inhibitory component is 1-1.8:0.0032:0.06.

7. The composition for stabilizing cell-free nucleic acid and/or exosomal nucleic acid in blood as claimed in claim 1, wherein the composition for stabilizing cell-free nucleic acid and/or exosomal nucleic acid in blood is the second composition, and wherein the allantoin-formaldehyde condensation product is diazolidinyl urea, the first metal chelator is aurintricarboxylic acid, the metabolic inhibitory component is NaF, and the second metal chelator is ethylenediaminetetraacetic acid.

8. The composition for stabilizing cell-free nucleic acid and/or exosomal nucleic acid in blood as claimed in claim 7, wherein a weight ratio of the antiseptic component, the enzyme inhibitory component, the metabolic inhibitory component and the anticoagulant component is 1-1.8:0.016:0.3:1.2-5.8.

9. A stabilizing agent for cell-free nucleic acid and/or exosomal nucleic acid in blood, which is selected from a group consisting of the following agents (a) and (b):
(a) a first agent, which comprises:
(i) an antiseptic component, comprising:
an allantoin-formaldehyde condensation product;
(ii) an enzyme inhibitory component, comprising:
a first metal chelator;
(iii) a metabolic inhibitory component, comprising:
NaF or sodium azide; and
(iv) a solvent,
wherein in the first agent, a content of the antiseptic component is 5-15% (w/v), a content of the enzyme inhibitory component is 0.005-0.25% (w/v), and a content of the metabolic inhibitory component is 0.025-0.4% (w/v); and
(b) a second agent, which comprises:
(i) an antiseptic component, comprising:
an allantoin-formaldehyde condensation product;
(ii) an enzyme inhibitory component, comprising:
a first metal chelator;
(iii) a metabolic inhibitory component, comprising:
NaF or sodium aside;
(iv) an anticoagulant component, comprising:
a second metal chelator; and
(v) a solvent,
wherein in the second agent,
a content of the antiseptic component is 1-3% (w/v), a content of the enzyme inhibitory component is 0.001-0.05% (w/v), a content of the metabolic inhibitory component is 0.1-2% (w/v), and a content of the anticoagulant component is 0.5-8% (w/v).

10. The stabilizing agent for cell-free nucleic acid and/or exosomal nucleic acid in blood as claimed in claim 9, wherein the allantoin-formaldehyde condensation product comprises at least one of diazolidinyl urea and imidazolidinyl urea, the first metal chelator comprises aurintricarboxylic acid, the second metal chelator comprises at least one of ethylenediaminetetraacetic acid and citrate, and the solvent comprises water.

11. The stabilizing agent for cell-free nucleic acid and/or exosomal nucleic acid in blood as claimed in claim 9, wherein the stabilizing agent for cell-free nucleic acid and/or exosomal nucleic acid in blood is the first agent, and wherein the allantoin-formaldehyde condensation product is diazolidinyl urea, the first metal chelator is aurintricarboxylic acid, the metabolic inhibitory component is NaF, and the solvent is water.

12. The stabilizing agent for cell-free nucleic acid and/or exosomal nucleic acid in blood as claimed in claim 11, wherein in the first agent, a content of the antiseptic component is 5-9% (w/v), a content of the enzyme inhibitory component is 0.016% (w/v), and a content of the metabolic inhibitory component is 0.3% (w/v).

13. The stabilizing agent for cell-free nucleic acid and/or exosomal nucleic acid in blood as claimed in claim 9, wherein the stabilizing agent for cell-free nucleic acid and/or exosomal nucleic acid in blood is the second agent, and wherein the allantoin-formaldehyde condensation product is diazolidinyl urea, the first metal chelator is aurintricarboxylic acid, the metabolic inhibitory component is NaF, the second metal chelator is ethylenediaminetetraacetic acid, and the solvent is water.

14. The stabilizing agent for cell-free nucleic acid and/or exosomal nucleic acid in blood as claimed in claim 13, wherein in the second agent, a content of the antiseptic component is 1-1.8% (w/v), a content of the enzyme inhibitory component is 0.016% (w/v), a content of the metabolic inhibitory component is 0.3% (w/v), and a content of the anticoagulant component is 1.2-5.8% (w/v).

15. A collection kit for cell-free nucleic acid and/or exosomal nucleic acid in blood, comprising:
the stabilizing agent for cell-free nucleic acid and/or exosomal nucleic acid in blood as claimed in claim 9; and
a first container, for accommodating a blood sample.

16. The collection kit for cell-free nucleic acid and/or exosomal nucleic acid in blood as claimed in claim 15, wherein the allantoin-formaldehyde condensation product comprises at least one of diazolidinyl urea and imidazolidinyl urea, the first metal chelator comprises aurintricarboxylic acid, the second metal chelator comprises at least one of ethylenediaminetetraacetic acid and citrate, and the solvent comprises water.

17. The collection kit for cell-free nucleic acid and/or exosomal nucleic acid in blood as claimed in claim 15, wherein the stabilizing agent for cell-free nucleic acid and/or exosomal nucleic acid in blood is the first agent, and wherein the allantoin-formaldehyde condensation product is diazolidinyl urea, the first metal chelator is aurintricarboxylic acid, the metabolic inhibitory component is NaF, and the solvent is water.

18. The collection kit for cell-free nucleic acid and/or exosomal nucleic acid in blood as claimed in claim 15, wherein the stabilizing agent for cell-free nucleic acid and/or exosomal nucleic acid in blood is the second agent, and wherein the allantoin-formaldehyde condensation product is diazolidinyl urea, the first metal chelator is aurintricarboxylic acid, the metabolic inhibitory component is NaF, the second metal chelator is ethylenediaminetetraacetic acid, and the solvent is water.

19. The collection kit for cell-free nucleic acid and/or exosomal nucleic acid in blood as claimed in claim 15, wherein the stabilizing agent for cell-free nucleic acid and/or exosomal nucleic acid in blood is prefilled in the first container, and when the blood sample is obtained, the blood sample is directly loaded into the first container to mix with the stabilizing agent for cell-free nucleic acid and/or exosomal nucleic acid in blood.

20. The collection kit for cell-free nucleic acid and/or exosomal nucleic acid in blood as claimed in claim 15, wherein the stabilizing agent for cell-free nucleic acid and/or exosomal nucleic acid in blood is loaded in a second container, and when the blood sample is obtained, the blood sample is directly loaded into the first container, and the stabilizing agent for cell-free nucleic acid and/or exosomal nucleic acid in blood is removed from the second container and added to the first container to mix with the blood sample.


SEQUENCE LISTING (3 sequences)

SEQ ID NO: 1 - DNA, 19 bp, synthetic construct
  source 1..19: mol_type = other DNA; note = Forward primer for wild type exon 2
aggcacgagtaacaagctc

SEQ ID NO: 2 - DNA, 23 bp, synthetic construct
  source 1..23: mol_type = other DNA; note = Reverse primer for wild type exon 2
ccacctcacagttattgaacatc

SEQ ID NO: 3 - DNA, 24 bp, synthetic construct
  source 1..24: mol_type = other DNA; note = Probe for wild type exon 2
cacgcagttgggcacttttgaaga